(19) United States
(12) Patent Application Publication
(10) Pub. No.: US 2021/0133356 A1
(43) Pub. Date: May 6, 2021

(54) Anonymization of Medical Image Data

(71) Applicant: Siemens Healthcare GmbH, Erlangen (DE)

(72) Inventor: Tobias Lenich, Nürnberg (DE)

(21) Appl. No.: 17/081,237

(22) Filed: Oct. 27, 2020

(30) Foreign Application Priority Data
Oct. 30, 2019 (DE) 10 2019 216 745.4

(51) Int. Cl. G06F 21/62; G16H 30/20; G16H 30/40; G06N 20/00

(52) U.S. Cl. CPC G06F 21/6254; G06N 20/00; G16H 30/40; G16H 30/20

(57) ABSTRACT

A computer-implemented method for provides classified image features. A plurality of image features are identified in the medical image data. The plurality of image features are classified into patient-specific image features and non-patient-specific image features by applying to input data a trained model for identifying and classifying image features. The input data is based upon the medical image data, providing the classified image features. A computer-implemented method provides synthetic medical image data and different computer-implemented methods provide trained models for identifying and classifying image features, for classifying patient-specific image features, and for generating synthetic medical image data.

### RELATED CASE

This application claims the benefit of German Application 10 2019 216 745.4, filed on Oct. 30, 2019, which is hereby incorporated by reference in its entirety.

### FIELD

The embodiments relate to a computer-implemented method for providing classified image features, a computer-implemented method for providing synthetic medical image data, a computer-implemented method for providing a trained model for identifying and classifying image features, a computer-implemented method for providing a trained model for classifying patient-specific image features, a computer-implemented method for providing a trained model for generating synthetic medical image data, a computer-implemented method for providing a further trained model for generating synthetic medical image data, a computer-implemented method for providing a further trained model for classifying patient-specific image features, a provision apparatus for providing classified image features, a provision apparatus for providing synthetic medical image data, a medical imaging device, a training apparatus, a computer program product, and a computer-readable storage medium.

### BACKGROUND

Patient data and/or scan data from patients, in particular medical image data that can be processed and/or output by clinics and/or physician practices, should be anonymized securely and as completely as possible. Conventionally, it was often sufficient to remove data, in particular text data and/or metadata that describes the patient, for example, name and date of birth, from the scan data. The scan data can thereby be present, for example, in a DICOM format, wherein the text data and/or metadata which describe the patient are often contained in the DICOM header.

With increasing scan accuracy, modern 3D imaging methods and improved reconstruction algorithms, it has become possible to reconstruct phenotypic features of a patient based upon the scan data. Hereby, for example, in magnetic resonance mappings and/or X-ray images, a reconstruction of a skull, a face and/or other phenotypic features of the patient can take place.

These features which are suitable for identifying a patient can be regarded as biometric features. The features described above can be obvious to a person skilled in the art, so that it is often attempted suitably to prevent such a reconstruction.

With regard to the constantly growing demand for, in particular, clinical medical image data for training of algorithms based on machine learning (ML), the removal of biometric features in the medical image data has grown significantly in importance. The known ML algorithms can hereby create many further biometric features from the medical image data that are not immediately apparent to a person skilled in the art.

### SUMMARY AND DETAILED DESCRIPTION

It is therefore the object to enable a secure anonymization of medical image data while maintaining the diagnosability.

The achievement of the object is described below, both in relation to methods and apparatuses for providing classified image features and/or synthetic medical image data as well as in relation to methods and apparatuses for providing trained models (trained functions). Herein, features, advantages and alternative embodiments of data structures and/or functions in methods and apparatuses for providing classified image features and/or synthetic medical image data can be transferred to analogous data structures and/or functions for methods, systems, and apparatuses for providing trained models. Herein, analogous data structures can be characterized, in particular, by the use of the qualifier “training”. Furthermore, the trained models used in methods, systems, and apparatuses for providing classified image features and/or synthetic medical image data can, in particular, have been adapted and/or provided by methods, systems, and apparatuses for providing trained models.

The embodiments relates in a first aspect to a computer-implemented method for providing classified image features. Thereby, in a first act, medical image data is received. By applying to input data a trained model for identifying and classifying image features, a plurality of image features are identified in the medical image data and the plurality of image features are classified in patient-specific and non-patient-specific image features. Thereby, the input data is based upon the medical image data. Additionally, at least one parameter of the trained model for identifying and classifying image features is based upon a comparison of training identification parameters with comparative identification parameters and a comparison of training diagnostic parameters with comparative diagnostic parameters. Additionally, the classified image features are provided in a further act.

The reception of the medical image data can include, in particular, an acquisition and/or readout of a computer-readable data store and/or a reception from a data storage apparatus, for example, a database. Additionally, the medical image data can be provided by a provision apparatus of a medical imaging device for recording the medical image data.

The medical image data can have, for example, two-dimensional and/or three-dimensional image data, comprising a plurality of image points, in particular pixels and/or voxels. The medical image data can also map at least one examination region of an examination object. Thereby, the examination object can include, for example, a human and/or an animal patient. In addition, the medical image data can map a temporal sequence, for example, a change in the examination region of the examination object. Furthermore, the medical image data can be recorded by one or more, in particular, different medical imaging devices. Therein, the one or at least one of the plurality of medical imaging devices can be configured as an X-ray device and/or a C-arm X-ray device and/or a magnetic resonance system (MRT) and/or a computed tomography system (CT) and/or a sonography system and/or a positron emission tomography system (PET).

Furthermore, the medical image data can advantageously include metadata. Therein, the metadata can include information relating to recording parameters and/or operating parameters of the medical imaging device for recording the medical image data.

By applying to the received medical image data the trained model for identifying and classifying image features, a plurality of image features can be identified in the medical image data. Additionally, the identified plurality of image features can be classified into patient-specific image features and non-patient-specific image features.

The plurality of image features in the medical image data can include, for example, geometrical image features and/or anatomical image features. Additionally, the plurality of image features can include an, in particular, statistical image information item which maps a distribution of image values within the medical image data, for example, a histogram. The identification of the plurality of image features by applying the trained model can include, in particular, a localization and/or segmentation of the plurality of image features in the medical image data.

The classification of the identified plurality of image features can also include a differentiation and/or grouping of the plurality of image features into patient-specific and non-patient-specific image features. The patient-specific image features can include, in particular, the image features that enable an, in particular, unambiguous mapping onto the examination object. Additionally, the patient-specific image features can include, for example, biometric image features and/or diagnostic image features which enable an inference and/or an, in particular, unambiguous identification of the examination object. Furthermore, the non-patient-specific image features can include, for example, diagnostic and/or further anatomical and/or geometrical image features which do not enable an inference and/or an identification of the examination object. For example, a contrast, in particular, a ratio of image values can be classified as a non-patient-specific image feature. Additionally, a spatial contrast variation, for example, an edge along an anatomical structure can be identified as an anatomical image feature and classified as a patient-specific image feature. In particular, the patient-specific image features can include all the biometric image features which are identified in the medical image data. Biometric image features can include, for example, a spatial position information item and/or a spatial arrangement information item and/or a shape information item of at least one anatomical image feature. For example, a skull shape and/or a tumor surface and/or an organ surface and/or a spatial arrangement of a plurality of anatomical image features relative to one another can be classified as a patient-specific image feature, in particular, a biometric image feature.

The advantages and/or properties of a trained model described below correspond substantially to the advantages of the proposed trained model for identifying and classifying image features. Features, advantages or alternative embodiments mentioned herein can also be transferred to the other proposed trained models and vice versa.

The trained model can advantageously be trained by a machine learning method. In particular, the trained model can be a neural network, in particular, a convolutional neural network (CNN) or a network comprising a convolution layer.

The trained model maps input data onto output data. Herein, in particular, the output data can further depend upon one or more parameters of the trained model. The one or more parameters of the trained model can be determined and/or adapted by a training. The determination and/or the adaptation of the one or more parameters of the trained model can be based, in particular, upon a pair made from training input data and associated training output data, wherein the trained model is applied to the training input data to generate training mapping data. In particular, the determination and/or the adaptation can be based upon a comparison of the training mapping data and the training output data. In general, a trainable function, that is, a function with one or a plurality of parameters not yet adapted, can also be designated a trained model.

Other expressions for trained model are trained mapping rule, mapping rule with trained parameters, function with trained parameters, algorithm based upon artificial intelligence, machine learning algorithm. An example of a trained model is an artificial neural network whereby the edge weights of the artificial neural network correspond to the parameters of the trained model. In place of the expression “neural network”, the expression “neural net” can also be used. In particular, a trained model can also be a deep neural network (or deep artificial neural network). A further example of a trained model is a “support vector machine” and furthermore, in particular, other machine learning algorithms are usable as a trained model.

The trained model can be trained, in particular, by a back-propagation. Firstly, training mapping data can be determined by applying the trained model to training input data. Thereafter, a deviation between the training mapping data and the training output data can be ascertained by applying an error function to the training mapping data and the training output data. Additionally, at least one parameter, in particular, a weighting of the trained model, in particular, of the neural network can be iteratively adapted based upon a gradient of the error function with regard to the at least one parameter of the trained model. By this approach, the deviation between the training mapping data and the training output data can advantageously be minimized during the training of the trained model.

Advantageously, the trained model, in particular the neural network, has an input layer and an output layer. Thereby, the input layer can be configured for receiving input data. Additionally, the output layer can be configured to provide mapping data. Thereby, the input layer and/or the output layer can each include a plurality of channels, in particular, neurons.

Preferably, at least one parameter of the trained model for identifying and classifying image features is based upon a comparison of training identification parameters with comparative identification parameters and a comparison of training diagnostic parameters with comparative diagnostic parameters. Thereby, the training identification parameters, the training diagnostic parameters, the comparative identification parameters and/or the comparative diagnostic parameters can be determined as part of a proposed computer-implemented method for providing a trained model for identifying and classifying image features, which is explained later in the description.

Furthermore, the provision of the classified image features can include, in particular, a storage on a computer-readable storage medium and/or a display on a display apparatus and/or a transfer to a provision apparatus.

By this approach, a particularly robust and reliable identification and classification of the image features in the medical image data is enabled.

In a further advantageous embodiment of the proposed computer-implemented method for providing classified image features, the patient-specific image features can be classified into phenotypically expressed patient-specific image features and non-phenotypically expressed patient-specific image features by applying to input data a trained model for classifying patient-specific image features. Therein, the input data can be based upon the patient-specific image features. Additionally, at least one parameter of the trained model for classifying patient-specific image features can be based upon a comparison of phenotypically expressed patient-specific training image features with phenotypically expressed patient-specific comparative image features and a comparison of non-phenotypically expressed patient-specific training image features with non-phenotypically expressed patient-specific comparative image features. Additionally, the classified patient-specific image features can be provided.

The advantages and/or properties of a trained model described above correspond substantially to the advantages of the proposed trained model for classifying patient-specific image features. Features, advantages or alternative embodiments mentioned herein can also be transferred to the proposed trained model for classifying patient-specific image features and vice versa.

Advantageously, the classification of the patient-specific image features can include a differentiation and/or grouping of the patient-specific image features into phenotypically expressed patient-specific image features and non-phenotypically expressed patient-specific image features. Therein, the phenotypically expressed patient-specific image features can, in particular, include all the patient-specific image features which enable an, in particular, unambiguous identification and/or an inference regarding the examination object based upon a matching between the patient-specific image feature and a further image feature which is acquirable by an external observation of the examination object. Additionally, the non-phenotypically expressed patient-specific image features can, in particular, include all the patient-specific image features which are not acquirable by an external observation of the examination object. Therein, the phenotypically expressed patient-specific image features can include, for example, an information item relating to at least a part of a face and/or a body shape of the examination object. Additionally, the non-phenotypically expressed patient-specific image features can include, for example, a shape information item relating to an internal organ of the examination object.

Preferably, at least one parameter of the trained model for classifying patient-specific image features can be based upon a comparison of phenotypically expressed patient-specific training image features with phenotypically expressed patient-specific comparative image features and a comparison of non-phenotypically expressed patient-specific training image features with non-phenotypically expressed patient-specific comparative image features. Therein, the phenotypically expressed patient-specific training image features, the phenotypically expressed patient-specific comparative image features, the non-phenotypically expressed patient-specific training image features and/or the non-phenotypically expressed patient-specific comparative image features can be determined as part of a proposed computer-implemented method for providing a trained model for classifying patient-specific image features, which is explained later in the description.

Furthermore, the provision of the classified patient-specific image features can include, in particular, a storage on a computer-readable storage medium and/or a display on a display apparatus and/or a transfer to a provision apparatus.

By this approach, the classification of the patient-specific image features can advantageously be extended to the phenotypically expressed patient-specific image features acquirable, in particular, by an external observation of the examination object. Additionally, non-phenotypically expressed image features, in particular, patient-specific non-phenotypically expressed diagnostically relevant image features can be classified particularly reliably.

The embodiments relate in a second aspect to a computer-implemented method for providing synthetic medical image data. Thereby, in a first act, medical image data is received. Additionally, in a second act, classified image features are received by applying to the medical image data an embodiment of the proposed computer-implemented method for providing classified image features. Furthermore, in a third act, synthetic medical image data is generated by applying to input data a trained model for generating synthetic medical image data. Therein, the input data is based upon the patient-specific image features. Additionally, at least one parameter of the trained model for generating synthetic medical image data is based upon a comparison of synthetic medical training image data with synthetic medical comparative image data. In a further act, the synthetic medical image data is provided.

The reception of the medical image data can include, in particular, an acquisition and/or readout of a computer-readable data store and/or a reception from a data storage apparatus, for example, a database. Additionally, the medical image data can be provided by a provision apparatus of the medical imaging device for recording the medical image data.

Similarly thereto, the classified image features that are provided by applying an embodiment of the proposed method for providing classified image features can be received. Therein, the reception of the classified image features can include an acquisition and/or readout of a computer-readable data store and/or a reception from a data storage apparatus, for example, a database. Therein, the reception of classified image features can preferably include patient-specific and non-patient-specific image features. Additionally, the received patient-specific image features can be further classified into phenotypically expressed patient-specific image features and non-phenotypically expressed patient-specific image features.

The advantages and/or properties of a trained model as described above correspond substantially to the advantages of the proposed trained model for generating synthetic medical image data. Features, advantages or alternative embodiments mentioned herein can be transferred to the proposed trained model for generating synthetic medical image data and vice versa.

By applying the trained model for generating synthetic medical image data to the received patient-specific image features, the synthetic medical image data can be generated. Therein, at least one parameter of the trained model for generating synthetic medical image data is based upon a comparison of synthetic medical training image data with synthetic medical comparative image data. Therein, the synthetic medical training images and the synthetic medical comparative image data are determined as part of a proposed computer-implemented method for providing a trained model for generating synthetic medical image data, which is explained later in the description.

Advantageously, the synthetic medical image data has all the patient-specific image features. Therein, the synthetic medical image data advantageously includes a mapping of at least one extract of the examination region of the examination object. Therein, the synthetic medical image data can advantageously correspond in its image properties, for example, in contrast and/or dimensionality and/or image geometry, to the received medical image data. Additionally, the synthetic medical image data can be generated based upon at least one recording parameter of the medical imaging device for recording the medical image data or a further medical imaging device.

Furthermore, the provision of the synthetic medical image data can include, in particular, a storage on a computer-readable storage medium and/or a display on a display apparatus and/or a transfer to a provision apparatus.

By this approach, advantageously, an improved evaluation of the phenotypic expression of the patient-specific image features contained in the synthetic medical image data can be enabled.

The embodiments relate in a third aspect to a further computer-implemented method for providing synthetic medical image data. Therein, in a first act, medical image data is received. Additionally, in a second act, classified image features are received by applying to the medical image data an embodiment of the proposed computer-implemented method for providing classified image features. In a third act, the synthetic medical image data is generated by applying to input data a further trained model for generating synthetic image data. Therein, the input data is based upon the non-patient-specific image features and/or the non-phenotypically expressed patient-specific image features. Additionally, at least one parameter of the further trained model for generating synthetic medical image data is based upon a comparison of synthetic medical training image data with synthetic medical comparative image data. In a further act, the synthetic medical image data is provided.

The reception of the medical image data can include, in particular, an acquisition and/or readout of a computer-readable data store and/or a reception from a data storage apparatus, for example, a database. Additionally, the medical image data can be provided by a provision apparatus of the medical imaging device for recording the medical image data.

Similarly thereto, the classified image features that are provided by applying an embodiment of the proposed method for providing classified image features can be received. Therein, the reception of the classified image features can include an acquisition and/or readout of a computer-readable data store and/or a reception from a data storage apparatus, for example, a database. Therein, the received classified image features can preferably include patient-specific and non-patient-specific image features. Additionally, the received patient-specific image features can be further classified into phenotypically expressed patient-specific image features and non-phenotypically expressed patient-specific image features.

The advantages and/or properties of a trained model described above correspond substantially to the advantages of the proposed further trained model for generating synthetic medical image data. Features, advantages or alternative embodiments mentioned herein can be transferred to the proposed further trained model for generating synthetic medical image data and vice versa.

By applying the further trained model for generating synthetic medical image data to the received non-patient-specific image features and/or the non-phenotypically expressed patient-specific image features, the synthetic medical image data can be generated. Therein, at least one parameter of the further trained model for generating synthetic medical image data is based upon a comparison of synthetic medical training image data with synthetic medical comparative image data. Therein, the synthetic medical training image data and the synthetic medical comparative image data can be determined as part of a proposed computer-implemented method for providing a further trained model for generating synthetic medical image data, which is explained later in the description.

Advantageously, the synthetic medical image data has all the non-phenotypically expressed patient-specific and/or non-patient-specific image features. Therein, the synthetic medical image data advantageously includes a mapping of at least one extract of the examination region of the examination object. Therein, the synthetic medical image data can advantageously correspond in its image properties, for example, in contrast and/or dimensionality and/or image geometry, to the received medical image data. Additionally, the synthetic medical image data can be generated based upon at least one recording parameter of the medical imaging device for recording the medical image data or a further medical imaging device.

Furthermore, the provision of the synthetic medical image data can include, in particular, a storage on a computer-readable storage medium and/or a display on a display apparatus and/or a transfer to a provision apparatus.

Hereby, advantageously, particularly secure anonymized synthetic medical image data can be provided which can be used as input data for further image processing algorithms and/or for training neural networks.

In a further advantageous embodiment of the proposed computer-implemented method for providing classified image features, by applying to the medical image data a proposed computer-implemented method for providing synthetic medical image data, synthetic medical image data can be received. In a further act, the patient-specific image features can be classified into phenotypically expressed patient-specific image features and non-phenotypically expressed patient-specific image features by applying to input data a further trained model for classifying patient-specific image features. Therein, the input data can be based upon the patient-specific image features and the synthetic medical image data. Additionally, at least one parameter of the further trained model for classifying patient-specific image features can be based upon a comparison of phenotypically expressed patient-specific training image features with phenotypically expressed patient-specific comparative image features and a comparison of non-phenotypically expressed patient-specific training image features with non-phenotypically expressed patient-specific comparative image features. In a further act, the classified patient-specific image features can be provided.

The reception of the synthetic medical image data which is provided by applying an embodiment of the proposed method for providing synthetic medical image data can include, in particular, an acquisition and/or readout of a computer-readable data store and/or a reception from a data storage apparatus, for example, a database. Additionally, the medical image data can be provided by a provision apparatus of the medical imaging device for recording the medical image data.

The advantages and/or properties described above of a trained model correspond substantially to the advantages of the proposed further trained model for classifying patient-specific image features. Features, advantages or alternative embodiments mentioned herein can also be transferred to the proposed further trained model for classifying patient-specific image features and vice versa.

Advantageously, the classification of the patient-specific image features by applying the further trained model for classifying patient-specific image features to the input data can include a differentiation and/or grouping of the patient-specific image features into phenotypically expressed patient-specific image features and non-phenotypically expressed patient-specific image features.

In that the input data of the further trained model for classifying patient-specific image features is based upon the patient-specific image features and the synthetic image data, the classification of the patient-specific image features can advantageously be enabled particularly precisely and/or taking account of an expression in the synthetic medical image data.

Preferably, at least one parameter of the further trained model for classifying patient-specific image features can be based upon a comparison of phenotypically expressed patient-specific training image features with phenotypically expressed patient-specific comparative image features and a comparison of non-phenotypically expressed patient-specific training image features with non-phenotypically expressed patient-specific comparative image features. Therein, the phenotypically expressed patient-specific training image features, the phenotypically expressed patient-specific comparative image features, the non-phenotypically expressed patient-specific training image features and/or the non-phenotypically expressed patient-specific comparative image features can be determined as part of a proposed computer-implemented method for providing a further trained model for classifying patient-specific image features, which is explained later in the description.

Furthermore, the provision of the classified patient-specific image features can include, in particular, a storage on a computer-readable storage medium and/or a display on a display apparatus and/or a transfer to a provision apparatus.

Hereby, a particularly reliable and secure classification in the synthetic medical image data of the patient-specific image features according to their phenotypic expression can be enabled. Therein, a phenotypic expression of the patient-specific image features can advantageously be taken into account, in particular, for further medical imaging modalities.

The embodiments relate in a fourth aspect to a computer-implemented method for providing a trained model for identifying and classifying image features. Therein, in a first act, medical training image data of a plurality of examination objects is received. By applying to input data the trained model for identifying and classifying image features, in a second act, a plurality of training image features are identified in the medical training image data and the plurality of training image features are classified in patient-specific training image features and non-patient-specific training image features. Therein, the input data is based upon the medical image data. Additionally, in a third act, training identification parameters and training diagnostic parameters are determined based upon the classified training image features. Therein, a training identification parameter and a training diagnostic parameter is determined for each of the classified training image features and/or for a combination of classified training image features. Furthermore, in a fourth act, a comparative identification parameter and a comparative diagnostic parameter is received for each of the examination objects. Therein, each comparative identification parameter includes an identification information item relating to one of the examination objects. Additionally, each comparative diagnostic parameter includes a diagnostic information item relating to one of the examination objects. In a fifth act, at least one parameter of the trained model for identifying and classifying image features is adapted based upon a comparison of the training identification parameters with the comparative identification parameters and of the training diagnostic parameters with the comparative diagnostic parameters. Additionally, the trained model is further provided for identifying and classifying image features in a sixth act.

The reception of the medical training image data of a plurality of examination objects can include, in particular, an acquisition and/or readout of a computer-readable data store and/or a reception from a data storage apparatus, for example, a database. Additionally, the medical training image data can be provided by a provision apparatus of at least one medical imaging device for recording the medical training image data. Advantageously, the medical training image data maps a plurality of, in particular different, examination objects. Additionally, the medical training image data can map a plurality of, in particular different, examination regions of the respective examination objects. Therein, the medical training image data can advantageously be recorded by one and/or more, in particular, different medical imaging devices. Therein, the medical training image data can be recorded, in particular, by a plurality of medical imaging devices of different imaging modalities and/or imaging methods.

The medical training image data can have, in particular, all the properties of the medical image data that has been described in relation to the computer-implemented method for providing classified image features and vice versa. In particular, the medical training image data can be medical image data. Additionally, the medical training image data can be simulated.

By applying the trained model for identifying and classifying to the input data that is based upon the medical training image data, advantageously, the plurality of training image features are identified in the medical training image data. Additionally, the plurality of training image features identified thereby can be classified into patient-specific training image features and non-patient-specific training image features.

Thereafter, a training identification parameter and a training diagnostic parameter can be determined for each of the classified training image features and/or for a combination of classified training image features. Therein, the training identification parameters can advantageously be determined by using an identification function, for example, a biometric and/or anatomical identification function, to the classified training image features. Advantageously, each of the training identification parameters can include an identification information item, for example, a biometric parameter that is suitable for identifying one of the plurality of examination objects. Furthermore, the training diagnostic parameters can be determined, for example, by determining a deviation of the classified training image features from an anatomical atlas and/or based upon artificial intelligence. Advantageously, each of the training diagnostic parameters can include a diagnostic information item relating to the respective classified training image feature and/or the respective combination of classified training image features. Therein, the diagnostic information can include, for example, a probability information item and/or an expression information item relating to a disease pattern and/or to an anatomical deviation relative to an, in particular healthy, anatomy.

Additionally, the training identification parameters and the training diagnostic parameters can be determined semi-automatically, for example, by annotation of the classified training image features. In particular, annotated classified training image features can be received.

The reception of the respective comparative identification parameter and of the respective comparative diagnostic parameter for each of the examination objects can include, in particular, an acquisition and/or readout of a computer-readable data store and/or a reception from a data storage apparatus, for example, a database.

Therein, the comparative identification parameters can advantageously include an identification information item relating in each case to one of the examination objects. For example, the identification information can include a biometric information item and/or an, in particular, photographic mapping relating to the respective examination object. Additionally, the comparative diagnostic parameters can advantageously include a diagnostic information item relating in each case to one of the examination objects. Therein, the diagnostic information can include, for example, a probability information item and/or an expression information item relating to a disease pattern of the respective examination object and/or to an anatomical deviation of the respective examination object relative to an, in particular healthy, anatomy.

Furthermore, at least one parameter of the trained model for identifying and classifying image features can be adapted based upon the comparison of the training identification parameters with the comparative identification parameters and of the training diagnostic parameters with the comparative diagnostic parameters. Therein, in particular, each of the training identification parameters can be compared with each of the comparative identification parameters. Additionally, in particular, each of the training diagnostic parameters can be compared with each of the comparative diagnostic parameters. The comparison between the training identification parameters and the comparative identification parameters and/or the comparison between the training diagnostic parameters and the comparative diagnostic parameters can advantageously be based upon a pattern recognition algorithm.

Since each of the training identification parameters and each of the training diagnostic parameters corresponds to one of the classified training image features and/or to a combination of classified training image features, by the comparison of the training identification parameters with the comparative identification parameters and the comparison of the training diagnostic parameters with the comparative diagnostic parameters, an exclusion of the classified training image features which do not enable identification of one of the examination objects and/or a diagnostic support can advantageously take place. Thereby, the identification of training image features can advantageously be improved by applying to the input data the trained model for identifying and classifying image features.

Additionally, the classification of the training image features in patient-specific and non-patient-specific training image features can advantageously be improved by applying the trained model for identifying and classifying image features, in particular, by the comparison of the training identification parameters with the comparative identification parameters.

The provision of the trained model for identifying and classifying image features can include, in particular, a storage on a computer-readable storage medium and/or a transfer to a provision apparatus.

Advantageously, with the proposed method for providing a trained model for identifying and classifying image features, a trained model for identifying and classifying image features can be provided which can be used in the computer-implemented method for providing classified image features.

According to a further advantageous embodiment of the computer-implemented method for providing a trained model for classifying patient-specific image features, at least one training recording parameter can be determined based upon the classified training image features. Additionally, at least one comparative recording parameter relating to the medical training image data of a plurality of examination objects can be received. Therein, the comparative recording parameter can advantageously include an information item relating to an operating parameter of the medical imaging device for recording the medical training image data and/or an information item relating to a recording geometry of the medical training image data. Therein, the at least one parameter of the trained model for identifying and classifying image features can, in particular, additionally be based upon a comparison of the at least one training recording parameter with the at least one comparative recording parameter. Thereby, the training image features can advantageously be classified as non-patient-specific training image features which are evoked by a unique recording parameter, in particular, within the medical training image data of a plurality of examination objects.

The embodiments relate in a fifth aspect to a computer-implemented method for providing a trained model for classifying patient-specific image features. Therein, in a first act, medical training image data of a plurality of examination objects is received. In a second act, classified training image features are received by applying to the medical training image data a proposed computer-implemented method for providing classified image features. Therein, the classified image features are provided as the classified training image features and the patient-specific image features are provided as patient-specific training image features. In a third act, the patient-specific training image features are classified into phenotypically expressed comparative image features and non-phenotypically expressed comparative image features by applying an, in particular, biometric identification function to the patient-specific training image features. Additionally, the patient-specific training image features are classified in a fourth act, by applying to input data the trained model for classifying patient-specific image features, into phenotypically expressed patient-specific training image features and non-phenotypically expressed training image features. Therein, the input data is based upon the patient-specific training image features.

Furthermore, at least one parameter of the trained model for classifying patient-specific image features is adapted in a fifth act, based upon a comparison of the phenotypically expressed patient-specific training image features with the phenotypically expressed patient-specific comparative image features and a comparison of the non-phenotypically expressed patient-specific training image features with the non-phenotypically expressed patient-specific comparative image features. In a sixth act, the trained model is provided for classifying patient-specific image features.

The medical training image data can have, in particular, all the properties of the medical training image data that has been described in relation to the computer-implemented method for providing a trained model for identifying and classifying image features and vice versa. In particular, the medical training image data can be medical image data.

The reception of the medical training image data of a plurality of examination objects can include, in particular, an acquisition and/or readout of a computer-readable data store and/or a reception from a data storage apparatus, for example, a database. Additionally, the medical training image data can be provided by a provision apparatus of at least one medical imaging device for recording the medical training image data. Furthermore, the medical training image data can be simulated.

Similarly thereto, the classified training image features that are provided by applying an embodiment of the proposed method for providing classified image features can be received. Therein, the reception of the classified training image features can include an acquisition and/or readout of a computer-readable data store and/or a reception from a data storage apparatus, for example, a database. The classified training image features can have, in particular, all the properties of the classified image features that have been described in relation to the computer-implemented method for providing classified image features and vice versa. In particular, the classified training image features can be classified image features. Therein, the received classified training image features can preferably include patient-specific and non-patient-specific training image features. Advantageously, the classified image features are provided as classified training image features. Therein, the patient-specific image features can advantageously be provided as patient-specific training image features.

Advantageously, the patient-specific training image features can be classified by applying the, in particular biometric, identification function to the patient-specific training image features, into phenotypically expressed patient-specific comparative image features and non-phenotypically expressed patient-specific comparative image features. Advantageously, the classification of the patient-specific training image features can include a differentiation and/or grouping of the patient-specific training image features into phenotypically expressed patient-specific comparative image features and non-phenotypically expressed patient-specific comparative image features. Additionally, the patient-specific training image features can be classified semi-automatically, for example, by annotation of the patient-specific training image features. In particular, annotated patient-specific training image features can be received.

In particular, by applying the identification function to the patient-specific training image features for each of the patient-specific training image features and/or for a combination of patient-specific training image features, a probability value can be determined that evaluates a phenotypic expression of the respective patient-specific training image feature. Herein, in particular, a spatial positioning, for example, a spatial position and/or alignment of the respective patient-specific training image feature can be taken into account based upon the medical training image data. Additionally, tissue parameters of tissue surrounding the respective patient-specific training image feature, for example, density information can advantageously be taken into account in the classification of the patient-specific training image features. In particular, the identification function can be applied to the patient-specific training image features and additionally to the medical training image data.

For example, an external observation of the respective examination object can be simulated by applying the identification function to the patient-specific training image features. Thereby, for example, algorithms known from the prior art for pattern recognition and/or biometric identification algorithms, in particular, for face recognition and/or based upon artificial intelligence, for acquiring the respective patient-specific training image feature through the simulated external observation of the respective examination object are applied. Provided a patient-specific training image feature is acquirable, in particular, by a simulated external observation of the respective examination object, the patient-specific training image feature can be classified by the identification function as a phenotypically expressed patient-specific comparative image feature. In particular, the classification of the patient-specific training image features can include, through the application of the identification function, a matching of the patient-specific training image features with, in particular known, phenotypically expressed biometric features. Provided a patient-specific training image feature is identified as a phenotypically expressed biometric feature, the patient-specific training image feature can be classified as a phenotypically expressed patient-specific comparative image feature.

Additionally, a patient-specific training image feature which is or could be acquirable under certain circumstances by an external observation of the respective examination object can be assigned a higher probability value than a patient-specific training image feature which is not acquirable by external observation of the respective examination object. For example, patient-specific training image features can be acquirable, in particular only, by observation of the examination object by a camera system, in particular, in a specific light wavelength range. Furthermore, patient-specific training image features acquirable by a camera system can enable an identification of the respective examination object, for example, by applying an artificial intelligence to the acquired patient-specific training image features. For this, the identification function can assign a high probability value to the respective patient-specific training image feature, so that this patient-specific training image feature can be classified as a phenotypically expressed comparative image feature.

The classification of patient-specific training image features by applying the identification function can advantageously be based upon a threshold value in relation to the probability value in relation to the acquirability of the respective patient-specific training image feature through external observation of the respective examination object.

Additionally, the classification of the training image features into phenotypically expressed patient-specific training image features and non-phenotypically expressed patient-specific training image features can advantageously be improved by applying the trained model for classifying patient-specific image features by the comparison of the phenotypically expressed patient-specific training image features with the phenotypically expressed patient-specific comparative image features and the comparison of the non-phenotypically expressed patient-specific training image features with the non-phenotypically expressed patient-specific comparative image features. In particular, each of the phenotypically expressed patient-specific training image features can be compared with each of the phenotypically expressed und non-phenotypically expressed comparative image features. Additionally, each of the non-phenotypically expressed patient-specific training image features can be compared with each of the phenotypically expressed und non-phenotypically expressed comparative image features.

The provision of the trained model for classifying patient-specific image features can include, in particular, a storage on a computer-readable storage medium and/or a transfer to a provision apparatus.

Advantageously, with the proposed method for providing a trained model for classifying patient-specific image features, a trained model for classifying patient-specific image features can be provided which can be used in the computer-implemented method for providing classified image features.

The embodiments relate in a sixth aspect to a computer-implemented method for providing a trained model for generating synthetic medical image data. Therein, in a first act, medical training image data of a plurality of examination objects is received. In a second act, classified training image features are received by applying to the medical training image data a proposed computer-implemented method for providing classified image features. Therein, the classified image features are provided as the classified training image features and the patient-specific image features are provided as patient-specific training image features. Additionally, in a third act, synthetic medical comparative image data is generated by applying a reconstruction function to the patient-specific training image features. In a fourth act, synthetic medical image data is generated by applying to input data the trained model for generating synthetic medical image data. Therein, the input data is based upon the patient-specific training image features. In a fifth act, at least one parameter of the trained model for generating synthetic medical image data is adapted based upon a comparison of the synthetic medical comparative image data with the synthetic medical training image data. In a sixth act, the trained model is provided for generating synthetic medical image data.

The medical training image data can have, in particular, all the properties of the medical training image data that has been described in relation to the computer-implemented method for providing a trained model for identifying and classifying image features and vice versa. In particular, the medical training image data can be medical image data.

The reception of the medical training image data of a plurality of examination objects can include, in particular, an acquisition and/or readout of a computer-readable data store and/or a reception from a data storage apparatus, for example, a database. Additionally, the medical training image data can be provided by a provision apparatus of at least one medical imaging device for recording the medical training image data. Furthermore, the medical training image data can be simulated.

Similarly thereto, the classified training image features that are provided by applying an embodiment of the proposed method for providing classified image features can be received. Therein, the reception of the classified training image features can include an acquisition and/or readout of a computer-readable data store and/or a reception from a data storage apparatus, for example, a database. The classified training image features can have, in particular, all the properties of the classified image features that have been described in relation to the computer-implemented method for providing classified image features and vice versa. In particular, the classified training image features can be classified image features. Therein, the received classified training image features can preferably include patient-specific and non-patient-specific training image features. Advantageously, the classified image features are provided as classified training image features. Therein, the patient-specific image features can advantageously be provided as patient-specific training image features.

Advantageously, the synthetic medical comparative image data can be generated, in particular, reconstructed by applying the reconstruction function to the patient-specific training image features. Therein, the reconstruction function can advantageously be configured to generate the synthetic medical comparative image data based upon the patient-specific training image features. Advantageously, the synthetic medical comparative image data for each of the examination objects can include a synthetic medical comparative individual image. Therein, the synthetic medical comparative individual images can be, for example, two-dimensional and/or three-dimensional. Therein, the synthetic medical comparative individual images can advantageously include, in particular, two-dimensional and/or three-dimensional, mapping of at least one extract of the examination region of the respective examination object.

Additionally, the reconstruction function can include a transform rule, for example, for a Fourier transform and/or a Radon transform, and/or an interpolation rule and/or an extrapolation rule for reconstructing the synthetic medical comparative image data. Advantageously, the reconstruction function can include a rule for reconstruction based upon incomplete input data, in particular, the patient-specific training image features. Additionally, the generation of the synthetic medical comparative image data by applying the reconstruction function can include an interpolation and/or extrapolation and/or transformation of the patient-specific training image features, in particular, based upon an anatomical information item and/or a recording parameter. Additionally, the synthetic medical comparative image data can also be generated based upon at least one recording parameter of the medical imaging device for recording the medical training image data or of a further medical imaging device.

Advantageously, the synthetic medical comparative image data has all the patient-specific training image features. Therein, the synthetic medical comparative image data can advantageously correspond in its image properties, for example, in contrast and/or dimensionality and/or image geometry, to the received medical training image data.

Advantageously, the synthetic medical training image data that is generated by applying the trained model for generating synthetic medical image data to the input data which is based upon the patient-specific training image features, can be improved by the comparison with the synthetic medical comparative image data. Advantageously, the synthetic medical training image data for each of the examination objects can include a synthetic medical training individual image. Therein, the synthetic medical training individual images can be, for example, two-dimensional and/or three-dimensional. Additionally, the comparison of the synthetic medical training image data with the synthetic medical comparative image data image can take place image point by image point, in particular, pixel-by-pixel and/or voxel-by-voxel. Additionally, the comparison can take place between the synthetic medical training individual images and the synthetic medical comparative individual images which correspond to a common examination object.

The provision of the trained model for generating synthetic medical image data can include a storage on a computer-readable storage medium and/or a transfer to a provision apparatus.

Advantageously, with the proposed method for providing a trained model for generating synthetic medical image data, a trained model for generating synthetic medical image data can be provided that can be used in the computer-implemented method for providing synthetic medical image data.

The embodiments relate in a seventh aspect to a computer-implemented method for providing a further trained model for generating synthetic medical image data. Therein, in a first act, medical training image data of a plurality of examination objects is received. Additionally, in a second act, classified training image features are received by applying to the medical training image data a proposed computer-implemented method for providing classified image features. Therein, the classified image features are provided as the classified training image features, the non-patient-specific image features as non-patient-specific training image features and/or the non-phenotypically expressed patient-specific image features as non-phenotypically expressed training image features. Additionally, in a third act, synthetic medical comparative image data is generated by applying a reconstruction function to the non-phenotypically expressed patient-specific training image features and/or the non-patient-specific training image features.

Furthermore, synthetic medical training image data is generated in a fourth act by applying to input data the further trained model for generating synthetic medical image data. Therein, the input data is based upon the non-phenotypically expressed patient-specific training image features and/or the non-patient-specific training image features. In a fifth act, at least one parameter of the further trained model for generating synthetic medical image data is adapted based upon a comparison of the synthetic medical comparative image data with the synthetic medical training image data. In a sixth act, the further trained model is provided for generating synthetic medical image data.

The medical training image data can have, in particular, all the properties of the medical training image data that has been described in relation to the computer-implemented method for providing a trained model for identifying and classifying image features and vice versa.

The reception of the medical training image data of a plurality of examination objects can include, in particular, an acquisition and/or readout of a computer-readable data store and/or a reception from a data storage apparatus, for example, a database. Additionally, the medical training image data can be provided by a provision apparatus of at least one medical imaging device for recording the medical training image data. Furthermore, the medical training image data can be simulated.

Similarly thereto, the classified training image features that are provided by applying an embodiment of the proposed method for providing classified image features can be received. Therein, the reception of the classified training image features can include an acquisition and/or readout of a computer-readable data store and/or a reception from a data storage apparatus, for example, a database. The classified training image features can have, in particular, all the properties of the classified image features that have been described in relation to the computer-implemented method for providing classified image features and vice versa. Additionally, the classified training image features can have, in particular, all the properties of the classified patient-specific image features that have been described in relation to the computer-implemented method for providing classified patient-specific image features and vice versa.

Additionally, the classified training image features can be classified image features. Therein, the received classified training image features can preferably include patient-specific and non-patient-specific training image features. Advantageously, the classified image features are provided as classified training image features. Therein, the patient-specific image features can advantageously be provided as patient-specific training image features. Furthermore, the patient-specific image features can be classified into phenotypically expressed patient-specific image features and non-phenotypically expressed patient-specific image features. Therein, the non-phenotypically expressed patient-specific image features can advantageously be provided as non-phenotypically expressed patient-specific training image features.

Advantageously, the synthetic medical comparative image data can be generated, in particular reconstructed, by applying the reconstruction function to the non-phenotypic patient-specific training image features. Therein, the reconstruction function can advantageously be configured to generate the synthetic medical comparative image data based upon the non-patient-specific training image features and/or the non-phenotypically expressed training image features. Advantageously, the synthetic medical comparative image data for each of the examination objects can include a synthetic medical comparative individual image in each case. Therein, the synthetic medical comparative individual images can be, for example, two-dimensional and/or three-dimensional. Therein, the synthetic medical comparative individual images can advantageously include an, in particular, two-dimensional and/or three-dimensional, mapping of at least one extract of the examination region of the respective examination object.

Additionally, the reconstruction function can include a transform rule, for example, for a Fourier transform and/or a Radon transform, and/or an interpolation rule and/or an extrapolation rule for reconstructing the synthetic medical comparative image data. Advantageously, the reconstruction function can include a rule for reconstruction based upon incomplete input data, in particular, the non-patient-specific training image features and/or the non-phenotypically expressed patient-specific training image features. Additionally, the generation of the synthetic medical comparative image data by applying the reconstruction function can include an interpolation and/or extrapolation and/or transformation of the non-patient-specific training image features and/or the non-phenotypically expressed patient-specific training image features, in particular, based upon an anatomical information item and/or a recording parameter. Additionally, the synthetic medical comparative image data can also be generated based upon at least one recording parameter of the medical imaging device for recording the medical training image data or of a further medical imaging device.

Advantageously, the synthetic medical comparative image data has all the non-patient-specific training image features and/or all the non-phenotypically expressed patient-specific training image features. Therein, the synthetic medical comparative image data can advantageously correspond in its image properties, for example, in contrast and/or dimensionality and/or image geometry, to the received medical training image data.

Advantageously, the synthetic medical training image data which is generated by applying to input data the further trained model for generating synthetic medical image data, which input data is based upon the non-patient-specific training image features and/or the non-phenotypically expressed patient-specific training image features, can be improved by the comparison with the synthetic medical comparative image data. Advantageously, the synthetic medical training image data for each of the examination objects can include a synthetic medical training individual image. Therein, the synthetic medical training individual images can be, for example, two-dimensional and/or three-dimensional. Additionally, the comparison of the synthetic medical training image data with the synthetic medical comparative image data can take place image point by image point, in particular, pixel-by-pixel and/or voxel-by-voxel. Additionally, the comparison can take place between the synthetic medical training individual images and the synthetic medical comparative individual images which correspond to a common examination object.

The provision of the further trained model for generating synthetic medical image data can include, in particular, a storage on a computer-readable storage medium and/or a transfer to a provision apparatus.

Advantageously, with the proposed method for providing a further trained model for generating synthetic medical image data, a further trained model for generating synthetic medical image data can be provided which can be used in the computer-implemented method for providing synthetic medical image data.

The embodiments relate in an eighth aspect to a computer-implemented method for providing a further trained model for classifying patient-specific image features. Therein, in a first act, medical training image data of a plurality of examination objects is received. Additionally, synthetic medical training image data is received by applying to the medical training image data a proposed computer-implemented method for providing synthetic medical image data. Therein, the synthetic medical image data is provided as the synthetic medical training image data and the patient-specific image features are provided as patient-specific training image features. In a second act, the patient-specific training image features are classified into phenotypically expressed patient-specific comparative image features and non-phenotypically expressed patient-specific comparative image features by applying a further, in particular, biometric identification function to the patient-specific training image features and the synthetic medical training image data. In a third act, the patient-specific training image features are classified into phenotypically expressed patient-specific training image features and non-phenotypically expressed training image features by applying to input data the further trained model for classifying patient-specific image features. Therein, the input data is based upon the patient-specific training image features and the synthetic medical training image data. In a fourth act, at least one parameter of the further trained model for classifying patient-specific image features is adapted, based upon a comparison of the phenotypically expressed patient-specific training image features with the phenotypically expressed patient-specific comparative image features and a comparison of the non-phenotypically expressed patient-specific training image features with the non-phenotypically expressed patient-specific comparative image features. In a fifth act, the further trained model is provided for classifying patient-specific image features.

The medical training image data can have, in particular, all the properties of the medical training image data that has been described in relation to the computer-implemented method for providing a trained model for identifying and classifying image features and vice versa. In particular, the medical training image data can be medical image data.

The reception of the medical training image data of a plurality of examination objects can include, in particular, an acquisition and/or readout of a computer-readable data store and/or a reception from a data storage apparatus, for example, a database. Additionally, the medical training image data can be provided by a provision apparatus of at least one medical imaging device for recording the medical training image data. Furthermore, the medical training image data can be simulated.

Similarly thereto, the synthetic medical training image data that is provided by applying to the medical training image data an embodiment of the proposed method for providing synthetic medical image data can be received. Therein, the reception of the synthetic medical training image data can include an acquisition and/or readout of a computer-readable data store and/or a reception from a data storage apparatus, for example, a database. The synthetic medical training image data can have, in particular, all the properties of the synthetic medical image data that have been described in relation to the computer-implemented method for providing synthetic medical image data and vice versa. In particular, the synthetic medical training image data can be synthetic medical image data. Advantageously, the synthetic medical training image data for each of the plurality of examination objects can include an, in particular, two-dimensional and/or three-dimensional training individual image.

Furthermore, the classified image features received during the application of the proposed method for providing synthetic medical image data can be provided as classified training image features. Therein, the classified training image features can preferably include patient-specific and non-patient-specific training image features. Additionally, the patient-specific image features can advantageously be provided as patient-specific training image features.

Advantageously, the patient-specific training image features can be classified by applying the further, in particular biometric, identification function to the patient-specific training image features and the synthetic medical training image data, into phenotypically expressed patient-specific comparative image features and non-phenotypically expressed patient-specific comparative image features. Advantageously, the classification of the patient-specific training image features can include a differentiation and/or grouping of the patient-specific training image features into phenotypically expressed patient-specific comparative image features and non-phenotypically expressed patient-specific comparative image features. Additionally, the patient-specific training image features can be classified semi-automatically, for example, by annotation of the patient-specific training image features. In particular, annotated patient-specific training image features can be received. Therein, a phenotypic expression of the patient-specific training image features in the synthetic medical training image data can advantageously be evaluated, in particular, by an external observation.

In particular, by applying the further identification function to the patient-specific training image features and the synthetic medical training image data for each of the patient-specific training image features and/or for a combination of patient-specific training image features, a probability value can be determined which evaluates a phenotypic expression of the respective patient-specific training image feature in the synthetic medical training image data. Herein, in particular, a spatial positioning, for example, a spatial position and/or alignment of the respective patient-specific training image feature can be taken into account in the synthetic medical training image data. Additionally, tissue parameters of tissue surrounding the respective patient-specific training image feature, for example, a density information item can advantageously be taken into account in the classification of the patient-specific training image features.

For example, an external observation of the respective examination object can be simulated by applying the further identification function to the patient-specific training image features and the synthetic medical training image data. Thereby, for example, algorithms known from the prior art for pattern recognition and/or biometric identification algorithms, in particular, for face recognition and/or based upon artificial intelligence, for acquiring the respective patient-specific training image feature through the simulated external observation of the respective examination object in the training individual image of the synthetic medical training image data are applied. Provided a patient-specific training image feature is acquirable, in particular, by a simulated external observation of the respective examination object in the synthetic medical training image data, the patient-specific training image feature can be classified by the further identification function as a phenotypically expressed patient-specific comparative image feature.

The classification of patient-specific training image features by applying the further identification function can advantageously be based upon a threshold value in relation to the probability value in relation to the acquirability of the respective patient-specific training image feature through external observation of the synthetic medical training image data.

Additionally, the classification of the training image features into phenotypically expressed patient-specific training image features and non-phenotypically expressed patient-specific training image features can advantageously be improved by applying the further trained model for classifying patient-specific image features by the comparison of the phenotypically expressed patient-specific training image features with the phenotypically expressed patient-specific comparative image features and the comparison of the non-phenotypically expressed patient-specific training image features with the non-phenotypically expressed patient-specific comparative image features. In particular, each of the phenotypically expressed patient-specific training image features can be compared with each of the phenotypically expressed and non-phenotypically expressed comparative image features. Additionally, each of the non-phenotypically expressed patient-specific training image features can be compared with each of the phenotypically expressed and non-phenotypically expressed comparative image features.

The provision of the further trained model for classifying patient-specific image features can include, in particular, a storage on a computer-readable storage medium and/or a transfer to a provision apparatus.

Advantageously, with the proposed method for providing a further trained model for classifying patient-specific image features, a further trained model for classifying patient-specific image features can be provided which can be used in the computer-implemented method for providing classified image features.

The embodiments relate in a ninth aspect to a provision apparatus for providing classified image features, comprising a computer and an interface. Therein, the interface is configured for receiving medical image data. Additionally, the computer is configured for identifying a plurality of image features in the medical image data and for classifying the plurality of image features into patient-specific and non-patient-specific image features by applying to input data a trained model for identifying and classifying image features. Therein, the input data is based upon the medical image data. Furthermore, at least one parameter of the trained model for identifying and classifying image features is based upon a comparison of training identification parameters with comparative identification parameters and a comparison of training diagnostic parameters with comparative diagnostic parameters. Additionally, the interface is configured to provide the classified image features.

Such a provision apparatus for providing classified image features can be configured, in particular, to carry out the previously described methods according to the embodiment for providing classified image features, and their aspects. The provision apparatus for providing classified image features is configured to carry out these methods and their aspects in that the interface and the computing apparatus are configured to carry out the corresponding method acts.

The advantages of the proposed provision apparatus for providing classified image features substantially correspond to the advantages of the proposed computer-implemented method for providing classified image features. Features, advantages or alternative embodiments mentioned herein can also be transferred to the other claimed subject matter and vice versa.

The embodiments relate in a tenth aspect to a provision apparatus for providing synthetic medical image data, comprising a computer and an interface. Therein, the interface is configured for receiving medical image data. Additionally, the interface is configured for receiving classified image features by applying to the medical image data a proposed computer-implemented method for providing classified image features. Additionally, the computer is configured for generating the synthetic medical image data by applying to input data a trained model for generating synthetic medical image data. Therein, the input data is based upon the patient-specific image features. Additionally, at least one parameter of the trained model for generating synthetic medical image data is based upon a comparison of synthetic medical training image data with synthetic medical comparative image data. Additionally, the interface is configured to provide the synthetic medical image data.

Such a provision apparatus for providing synthetic medical image data can be configured, in particular, to carry out the previously described methods according to the embodiment for providing synthetic medical image data, and their aspects. The provision apparatus for providing synthetic medical image data is configured to carry out these methods and their aspects in that the interface and the computer are configured to carry out the corresponding method acts.

The advantages of the proposed provision apparatus for providing synthetic medical image data substantially correspond to the advantages of the proposed computer-implemented method for providing synthetic medical image data. Features, advantages or alternative embodiments mentioned herein can also be transferred to the other claimed subject matter and vice versa.

The embodiments relate in an eleventh aspect to a medical imaging device comprising a proposed provision apparatus for providing classified image features and/or for providing synthetic medical image data. Therein, the medical imaging device, in particular, the proposed provision apparatus is configured for carrying out a proposed computer-implemented method for providing classified image features and/or for providing synthetic medical image data. Therein, the medical imaging device can be configured, for example, as a medical X-ray device, in particular, a C-arm X-ray device and/or a computed tomography (CT) system and/or a magnetic resonance (MRT) system and/or a sonography system. Additionally, the medical imaging device can be configured for recording and/or for receiving and/or for providing the medical image data.

The medical imaging device can include, in particular, a display apparatus, for example, a display screen and/or a monitor that is configured to display information and/or graphical representations of information of the medical imaging device and/or the provision apparatus and/or further components. In particular, the display apparatus can be configured for the display of a graphical representation of the medical image data and/or the classified image features and/or the synthetic medical image data.

The advantages of the proposed medical imaging device substantially correspond to the advantages of the proposed computer-implemented method for providing classified image features and/or for providing synthetic medical image data. Features, advantages or alternative embodiments mentioned herein can also be transferred to the other claimed subject matter and vice versa.

The embodiments relate in a twelfth aspect to a training apparatus that is configured to carry out the above described computer-implemented method according to the embodiment for providing a trained model for identifying and classifying image features, and/or for providing a trained model for classifying patient-specific image features and/or for providing a trained model for generating synthetic medical image data and/or for providing a further trained model for generating synthetic medical image data and/or for providing a further trained model for classifying patient-specific image features, and their aspects. The training apparatus advantageously includes a training interface and a training computing apparatus. The training apparatus is configured to carry out these methods and their aspects in that the training interface and the training computing apparatus are configured to carry out the corresponding method acts. In particular, the training interface can be configured for receiving medical training image data and/or classified training image features and/or synthetic medical training image data. Additionally, the training interface can be configured to provide the trained model.

The embodiments relate in a thirteenth aspect to a computer program product with a computer program that is directly loadable into a memory store of a provision apparatus, having program portions in order to carry out all the acts of the computer-implemented method for providing classified image features and/or for providing synthetic medical image data when the program portions are executed by the provision apparatus; and/or which is directly loadable into a training memory store of a training apparatus, having program portions in order to carry out all the acts of the proposed method for providing a training function for identifying and classifying image features and/or for providing a trained model for classifying patient-specific image features and/or for providing a trained model for generating synthetic medical image data and/or for providing a further trained model for generating synthetic medical image data and/or for providing a further trained model for classifying patient-specific image features and/or one of its aspects, when the program portions are executed by the training apparatus.

The embodiments relate in a fourteenth aspect to a computer-readable storage medium on which program portions that are readable and executable by a provision apparatus (processor) are stored in order to carry out all the acts of the computer-implemented method for providing classified image features and/or for providing synthetic medical image data when the program portions are executed by the provision apparatus; and/or on which program portions that are readable and executable by a training apparatus are stored in order to carry out all the acts of the method for providing a training function for identifying and classifying image features and/or for providing a trained model for classifying patient-specific image features and/or for providing a trained model for generating synthetic medical image data and/or for providing a further trained model for generating synthetic medical image data and/or for providing a further trained model for classifying patient-specific image features and/or one of its aspects when the program portions are executed by the training apparatus.

The embodiments relate in a fifteenth aspect to a computer program or a computer-readable storage medium comprising a trained model for identifying and classifying image features, and/or for classifying patient-specific image features and/or for generating synthetic medical image data and/or a further trained model for generating synthetic medical image data and/or for classifying patient-specific image features by a proposed computer-implemented method or one of its aspects.

A realization largely through software has the advantage that provision apparatuses and/or training apparatuses already used to date can also easily be upgraded with a software update in order to operate in the manner according to the embodiment. Such a computer program product can include, where relevant, in addition to the computer program, further constituent parts, such as, for example, documentation and/or additional components as well as hardware components, for example, hardware keys (dongles, etc.) for using the software.

The present invention is defined by the following claims, and nothing in this section should be taken as a limitation on those claims. Any teaching for one type of claim (e.g., method) may be applicable to another type of claim (e.g., computer readable storage medium or system). Further aspects and advantages of the invention are discussed below in conjunction with the preferred embodiments and may be later claimed independently or in combination.

### BRIEF DESCRIPTION OF THE DRAWINGS

Exemplary embodiments of the invention are illustrated in the drawings and are described in greater detail below. In the different figures, the same reference characters are used for the same features. In the drawings:

FIGS. 1 and 2 show schematic representations of different embodiments of a proposed computer-implemented method for providing classified image features,

FIGS. 3 and 4 show schematic representations of different embodiments of a proposed method for generating synthetic medical image data,

FIG. 5 shows a schematic representation of a further embodiment of the proposed computer-implemented method for providing classified image features,

FIG. 6 shows a schematic representation of an embodiment of a proposed computer-implemented method for providing a trained model for identifying and classifying image features,

FIG. 7 shows a schematic representation of an embodiment of a proposed computer-implemented method for providing a trained model for classifying patient-specific image features,

FIG. 8 shows a schematic representation of an embodiment of a proposed computer-implemented method for providing a trained model for generating synthetic medical image data,

FIG. 9 shows a schematic representation of an embodiment of a proposed computer-implemented method for providing a further trained model for generating synthetic medical image data,

FIG. 10 shows a schematic representation of an embodiment of a proposed computer-implemented method for providing a further trained model for classifying patient-specific image features,

FIG. 11 shows an example schematic representation of a proposed provision apparatus,

FIG. 12 shows an example schematic representation of a proposed training apparatus,

FIG. 13 shows a schematic representation of a medical C-arm X-ray device as an example of a proposed medical imaging device.

### DETAILED DESCRIPTION

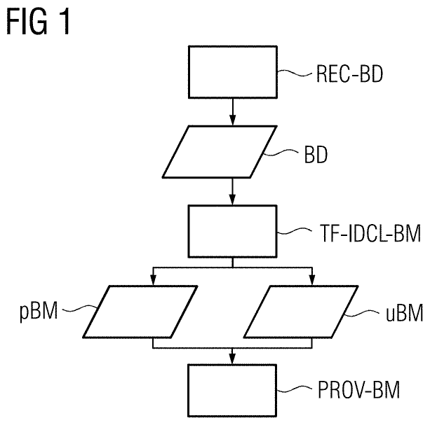

FIG. 1 is a schematic representation of an embodiment of the proposed computer-implemented method for providing classified image features. Therein, in a first act REC-BD, medical image data BD can be received. By applying to input data that is based upon the medical image data BD, a trained model for identifying and classifying image features TF-IDCL-BM, a plurality of image features can be identified in the medical image data BD. Therein, the plurality of image features can also be classified into patient-specific image features pBM and non-patient-specific image features uBM. Advantageously, at least one parameter of the trained model for identifying and classifying image features TF-IDCL-BM can be based upon a comparison of training identification parameters with comparative identification parameters and a comparison of training diagnostic parameters with comparative diagnostic parameters. In a further act PROV-BM, the classified image features pBM and uBM can be provided.

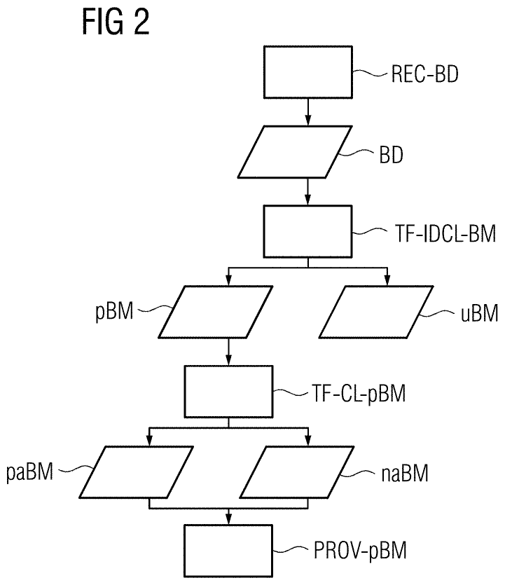

FIG. 2 is a schematic representation of a further embodiment of the proposed computer-implemented method for providing classified image features. Therein, the patient-specific image features pBM can be classified by applying to input data which is based upon patient-specific image features pBM, a trained model for classifying patient-specific image features TF-CL-pBM into phenotypically expressed patient-specific image features paBM and into non-phenotypically expressed patient-specific image features naBM. Therein, at least one parameter of the trained model for classifying patient-specific image features TF-CL-pBM can be based upon a comparison of phenotypically expressed patient-specific training image features with phenotypically expressed patient-specific comparative image features and a comparison of non-phenotypically expressed training image features with non-phenotypically expressed comparative image features. In a further act PROV-pBM, the classified patient-specific image features paBM and naBM can be provided.

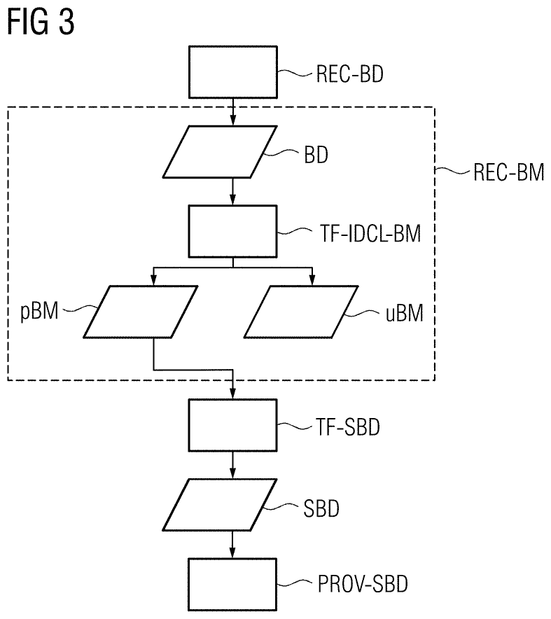

FIG. 3 is a schematic representation of an embodiment of a proposed computer-implemented method for generating synthetic medical image data. Therein, in a first act REC-BD, medical image data BD can be received. Additionally, in a second act REC-BM, classified image features pBM and uBM can be received by applying to the medical image data BD a proposed computer-implemented method for providing classified image features. In a next act, the synthetic medical image data SBD can be generated by applying to input data a trained model for generating synthetic medical image data TF-SBD. Therein, the input data can advantageously be based upon the patient-specific image features pBM. Additionally, at least one parameter of the trained model for generating synthetic medical image data TF-SBD can be based upon a comparison of synthetic medical training image data with synthetic medical comparative image data. In a further act PROV-SBD, the synthetic medical image data SBD can be provided.

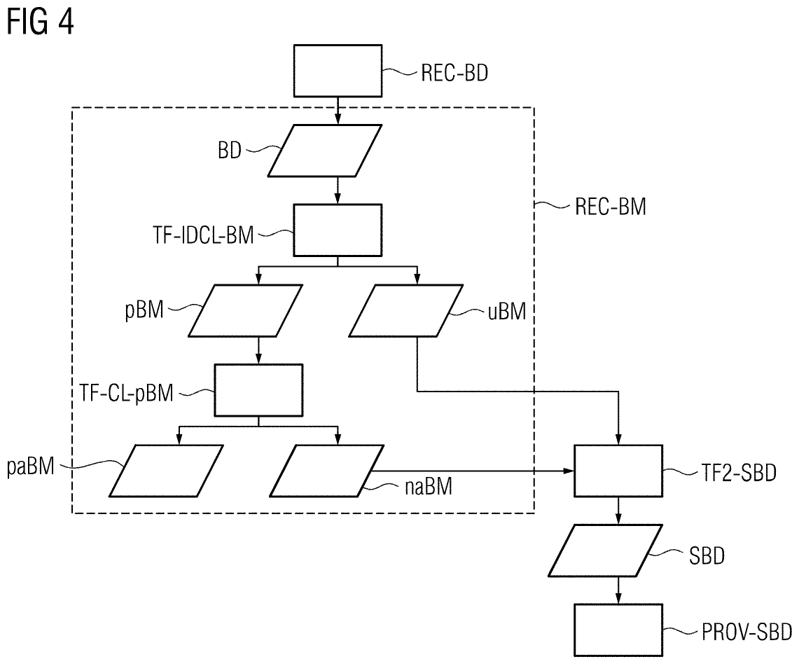

FIG. 4 is a schematic representation of a further embodiment of a proposed computer-implemented method for generating synthetic medical image data. Therein, in a first act REC-BD, medical image data BD can be received. Furthermore, classified image features can be received REC-BM by applying to the medical image data a proposed computer-implemented method for providing classified image features. Therein, the received classified image features can be classified into patient-specific image features pBM and non-patient-specific image features uBM. In addition, the patient-specific image features pBM can be configured in phenotypically expressed patient-specific image features paBM and in non-phenotypically expressed patient-specific image features naBM.

In a further act, the synthetic medical image data SBD can be generated by applying to input data a further trained model for generating synthetic medical image data TF2-SBD. Therein, the input data can advantageously be based upon the non-patient-specific image features uBM and the non-phenotypically expressed patient-specific image features naBM. Additionally, at least one parameter of the further trained model for generating synthetic medical image data TF2-SBD can be based upon a comparison of synthetic medical training image data with synthetic medical comparative image data. In a further act PROV-SBD, the synthetic medical image data SBD can be provided.

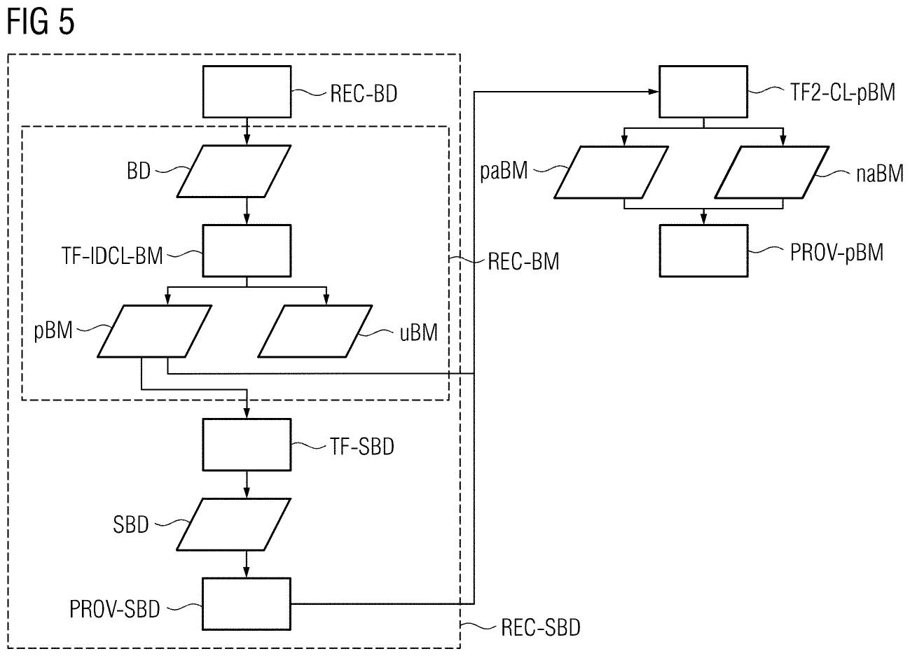

FIG. 5 is a schematic representation of a further embodiment of a proposed computer-implemented method for providing classified image features. Therein, in a further act REC-SBD, synthetic medical image data SBD can be received by applying to the medical image data BD a proposed computer-implemented method for generating synthetic medical image data. Furthermore, the patient-specific image features pBM can be classified by applying to input data a further trained model for classifying patient-specific image features TF2-CL-BM into phenotypically expressed patient-specific image features paBM and into non-phenotypically expressed patient-specific image features naBM. Therein, the input data can advantageously be based upon the patient-specific image features pBM and the synthetic medical image data SBD. Furthermore, at least one parameter of the further trained model for classifying patient-specific image features TF2-CL-BM can be based upon a comparison of phenotypically expressed patient-specific training image features with phenotypically expressed patient-specific comparative image features and a comparison of non-phenotypically expressed training image features with non-phenotypically expressed comparative image features. In a further act PROV-pBM, the classified patient-specific image features paBM and naBM can be provided.

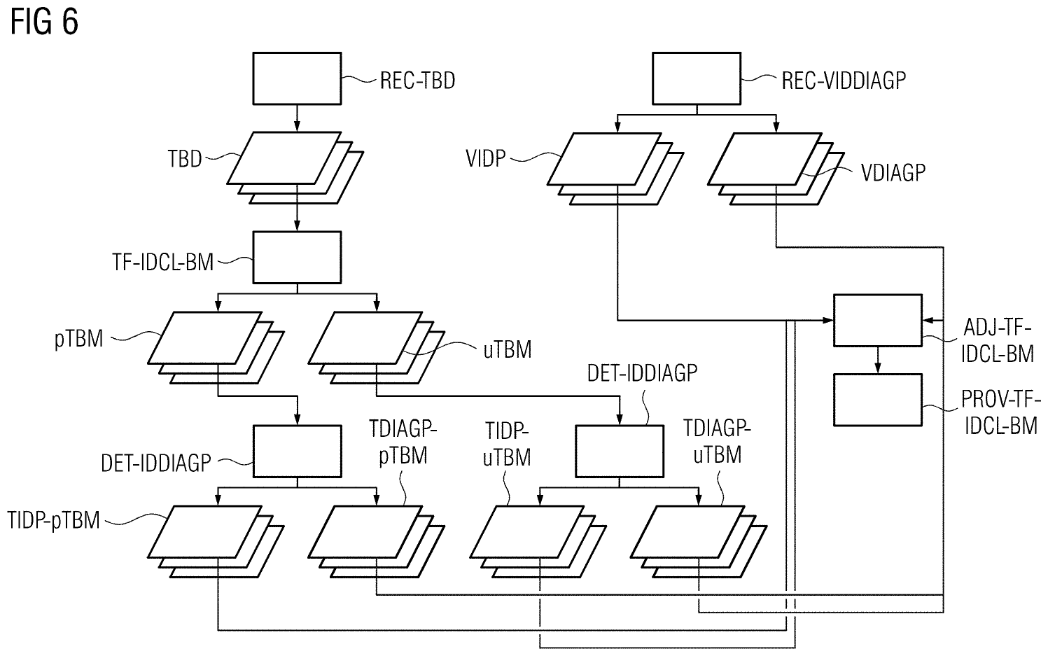

FIG. 6 is a schematic representation of an embodiment of a proposed computer-implemented method for providing a trained model for identifying and classifying image features TF-IDCL-BM. Therein, in a first act REC-TBD, medical training image data TBD of a plurality of examination objects can be received. In a second act, a plurality of training image features can be identified and classified in the medical training image data TBD by applying to input data the trained model for identifying and classifying image features TF-IDCL-BM. Therein, the input data can be based upon the medical training image data TBD. Furthermore, the plurality of image features can advantageously be classified into patient-specific training image features pTBM and non-patient-specific training image features uTBM. Thereafter, a training identification parameter TIDP-pTBM, TIDP-uTBM and a training diagnostic parameter TDIAGP-pTBM, TDIAGP-uTBM can be determined DET-IDDIAGP for each of the classified training image features pBM, uBM and/or for a combination of classified training image features pBM, uBM.

Furthermore, a comparative identification parameter VIDP and a comparative diagnostic parameter VDIAGP can be received REC-VIDDIAGP in each case for each of the examination objects. Therein, each comparative identification parameter VIDP can include an identification information item relating to one of the examination objects. Additionally, each comparative diagnostic parameter VDIAGP can include a diagnostic information item relating to one of the examination objects.

In a further act ADJ-TF-IDCL-BM, at least one parameter of the trained model for identifying and classifying image features TF-IDCL-BM can be adapted based upon a comparison of the training identification parameters TIDP-pTBM, TIDP-uTBM with the comparative identification parameters VIDP and a comparison of the training diagnostic parameters TDIAGP-pTBM, TDIAGP-uTBM with the comparative diagnostic parameters VDIAGP. Thereafter, the trained model for identifying and classifying image features TF-IDCL-BM can be provided PROV-TF-IDCL-BM.

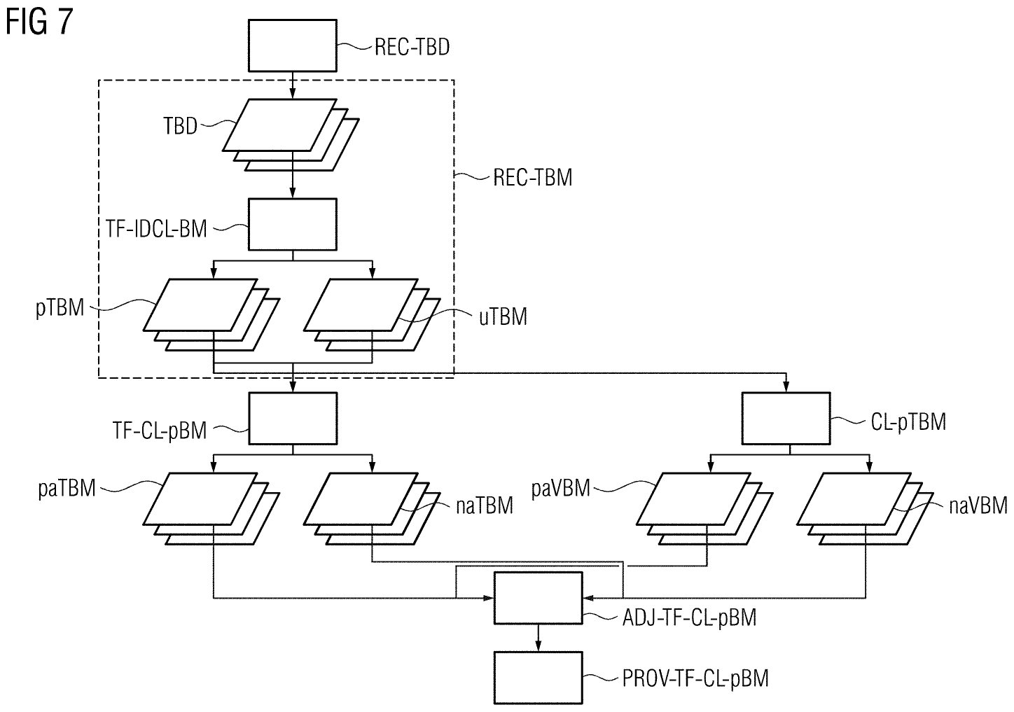

FIG. 7 is a schematic representation of an embodiment of a proposed computer-implemented method for providing a trained model for classifying patient-specific image features TF-CL-pBM. Therein, in a first act REC-TBD, medical training image data TBD of a plurality of examination objects can be received. Additionally, classified training image features pTBM and uTBM can be received REC-TBM by applying to the medical training image data TBD a proposed computer-implemented method for providing classified image features. Therein, the classified image features uBM and pBM can be provided as the classified training image features uTBM and pTBM. Thereafter, the patient-specific training image features pTBM can be classified by applying an, in particular biometric, identification function CL-pTBM to the patient-specific training image features pTBM, into phenotypically expressed patient-specific comparative image features paVBM and non-phenotypically expressed patient-specific comparative image features naVBM. Additionally, the patient-specific image features pTBM can be classified by applying to input data the trained model for classifying patient-specific image features TF-CL-pBM into phenotypically expressed patient-specific training image features paTBM and into non-phenotypically expressed patient-specific training image features naTBM. Therein, the input data can advantageously be based upon the patient-specific training image features pTBM.

In a further act ADJ-TF-CL-BM, at least one parameter of the trained model for classifying patient-specific image features TF-CL-pBM can be adapted based upon a comparison of the phenotypically expressed patient-specific training image features paTBM with the phenotypically expressed patient-specific comparative image features paVBM and a comparison of the non-phenotypically expressed patient-specific training image features naTBM with the non-phenotypically expressed patient-specific comparative image features naVBM. Thereafter, the trained model for classifying patient-specific image features TF-CL-pBM can be provided PROV-TF-CL-pBM.

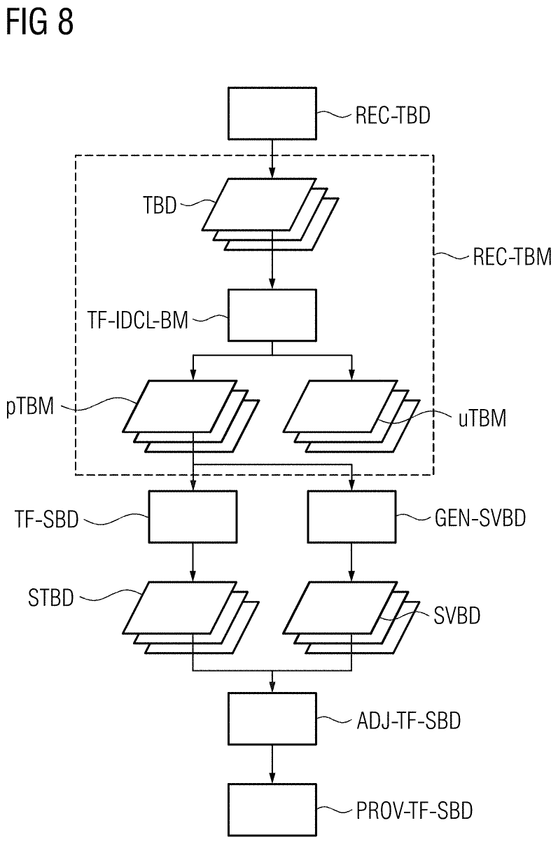

FIG. 8 is a schematic representation of an embodiment of the proposed computer-implemented method for providing a trained model for generating synthetic medical image data TF-SBD. Therein, in a first act REC-TBD, medical training image data TBD of a plurality of examination objects can be received. In a second act REC-TBM, classified training image features pTBM and uTBM can be received by applying to the training image data TBD a proposed computer-implemented method for providing classified image features. Therein, the classified image features pBM and uBM can be provided as the classified training image features pTBM and uTBM. In a third act, synthetic medical comparative image data SVBD can be generated GEN-SVBD by applying a reconstruction function to the patient-specific training image features pTBM. Additionally, synthetic medical training image data STBD can be generated by applying to input data the trained model for generating synthetic medical image data TF-SBD which is based upon the patient-specific training image features pTBM. Hereafter, at least one parameter of the trained model for generating synthetic medical image data TF-SBD can be adapted ADJ-TF-SBD based upon a comparison of the synthetic medical comparative image data SVBD with the synthetic medical training image data STBD. In a further act PROV-TF-SBD, the trained model can be provided for generating synthetic medical image data TF-SBD.

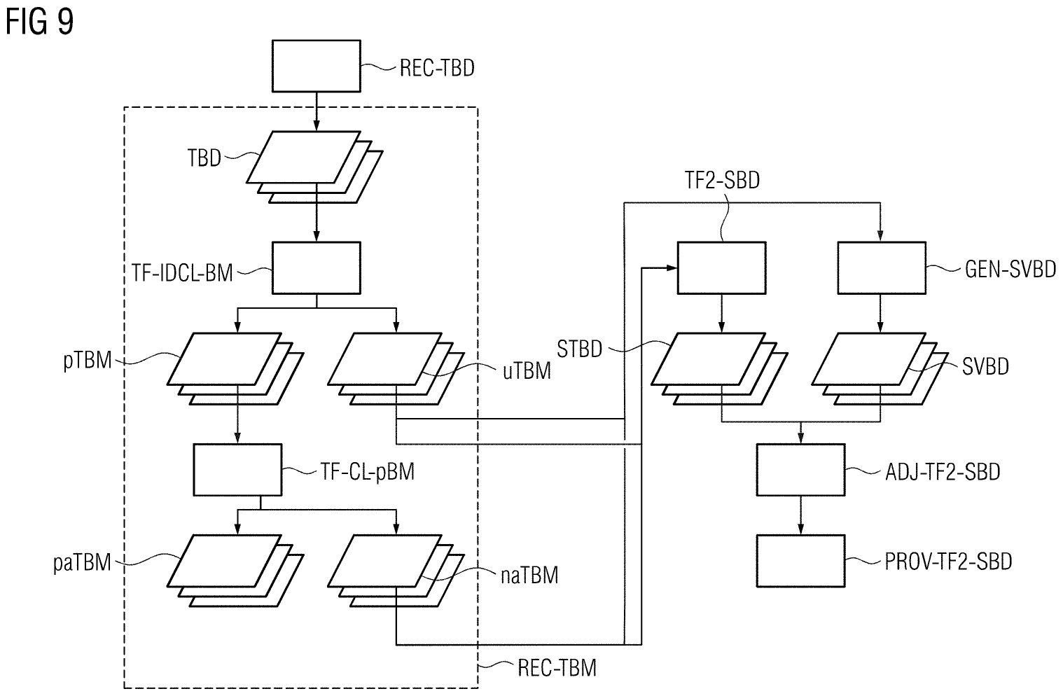

FIG. 9 is a schematic representation of an embodiment of the proposed computer-implemented method for providing a further trained model for generating synthetic medical image data TF2-SBD. Therein, in a first act REC-TBD, medical training image data TBD of a plurality of examination objects can be received. Additionally, in a second act REC-TBM, classified training image features can be received by applying to the medical training image data TBD a proposed computer-implemented method for providing classified image features. Therein, the classified image features pBM and uBM can be provided as the classified training image features pTBM and uTBM. Additionally, the phenotypically expressed patient-specific image features paBM can be provided as the phenotypically expressed patient-specific training image features paTBM. Similarly thereto, the non-phenotypically expressed patient-specific image features naBM can be provided as the non-phenotypically expressed patient-specific training image features naTBM.

In a further act GEN-SVBD, synthetic medical comparative image data SVBD can be generated by applying a further reconstruction function to the non-patient-specific training image features uTBM and the non-phenotypically expressed patient-specific training image features naTBM. Additionally, synthetic medical training image data STBD can be generated by applying to input data, which is based upon the non-patient-specific training image features uTBM and the non-phenotypically expressed patient-specific training image features naTBM, the further trained model for generating synthetic medical image data TF2-SBD.

Hereafter, at least one parameter of the further trained model for generating synthetic medical image data TF2-SBD can be adapted based upon a comparison of the synthetic medical training image data STBD with the synthetic medical comparative image data SVBD. In a further act PROV-TF2-SBD, the further trained model can be provided for generating synthetic medical image data TF2-SBD.

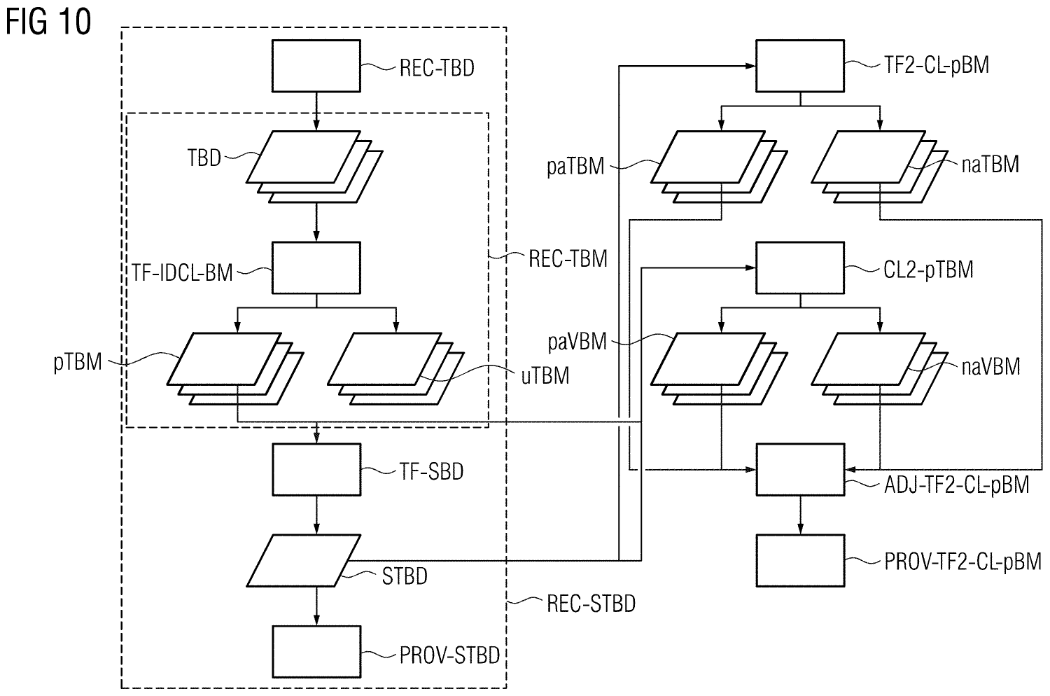

FIG. 10 is a schematic representation of an embodiment of the proposed computer-implemented method for providing a further trained model for classifying patient-specific image features TF2-CL-pBM. Therein, in a first act REC-TBD, medical training image data TBD of a plurality of examination objects can be received. In a second act REC-STBD, synthetic medical training image data STBD can be received by applying to the medical training image data TBD, in particular, to the patient-specific training image features pTBM, a proposed computer-implemented method for generating synthetic medical image data. Therein, the synthetic medical image data SBD is provided as the synthetic medical training image data STBD and the patient-specific image features pBM are provided as patient-specific training image features pTBM. In a third act, the patient-specific training image features pTBM can be classified, by applying a further, in particular biometric, identification function CL2-pTBM to the patient-specific training image features pTBM and the synthetic medical training image features STBD, into phenotypically expressed patient-specific comparative image features paVBM and non-phenotypically expressed patient-specific comparative image features naVBM. Additionally, the patient-specific training image features pTBM can be classified by applying to input data the further trained model for classifying patient-specific image features TF2-CL-pBM into phenotypically expressed patient-specific training image features paTBM and into non-phenotypically expressed patient-specific training image features naTBM. Therein, the input data can advantageously be based upon the patient-specific training image features pTBM and the synthetic medical training image data STBD.

Thereafter, at least one parameter of the further trained model for classifying patient-specific image features TF-CL-pBM can be adapted ADJ-TF2-CL-pBM based upon a comparison of the phenotypically expressed patient-specific training image features paTBM with the phenotypically expressed patient-specific comparative image features paVBM and a comparison of the non-phenotypically expressed patient-specific training image features naTBM with the non-phenotypically expressed patient-specific comparative image features naVBM. In a further act PROV-TF2-CL-pBM, the further trained model can be provided for classifying patient-specific image features TF2-CL-pBM.

FIG. 11 shows a provision apparatus PRVS comprising an interface IF, a computing unit or apparatus CU (computer) and a storage unit or apparatus MU (memory or database). Therein, the provision apparatus PRVS can be configured to provide classified image features PROV-BM and/or PROV-pBM. Therein, the interface IF can be configured for receiving medical image data BM. Additionally, the computing apparatus CU can be configured for identifying a plurality of image features in the medical image data BD and for classifying the plurality of image features into patient-specific image features pBM and non-patient-specific image features uBM by applying to input data a trained model for identifying and classifying image features TF-IDCL-BM. Therein, the input data can be based upon the medical image data BD. Additionally, the interface IF can be configured to provide the classified image features PROV-BM and/or PROV-pBM.

Such a provision apparatus PRVS for providing classified image features PROV-BM and/or PROV-pBM can be configured, in particular, to carry out the previously described methods according to the embodiment for providing classified image features, and their aspects. The provision apparatus PRVS for providing classified image features PROV-BM and/or PROV-pBM can be configured to carry out these methods and their aspects in that the interface IF and the computing apparatus CU are configured to carry out the corresponding method acts.

Furthermore, the provision apparatus PRVS can be configured to provide synthetic medical image data PROV-SBD. For this purpose, the interface IF can be configured for receiving classified image features by applying to the medical image data BD a proposed computer-implemented method for providing classified image features. Additionally, the computing apparatus CU is configured for generating the synthetic medical image data SBD by applying to input data the trained model for generating synthetic medical image data TF-SBD. Therein, the input data can be based upon the patient-specific image features. Additionally, the interface IF can be configured to provide the synthetic medical image data SBD.

Such a provision apparatus PRVS for providing synthetic medical image data PROV-SBD can be configured, in particular, to carry out the previously described methods according to the embodiment for providing synthetic medical image data, and their aspects. The provision apparatus PRVS for providing synthetic medical image data PROV-SBD can be configured to carry out these methods and their aspects in that the interface IF and the computing apparatus CU are configured to carry out the corresponding method acts.

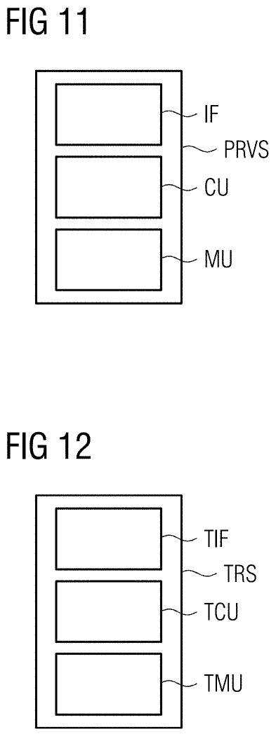

FIG. 12 shows a training apparatus TRS comprising a training interface TIF, a training computing unit or apparatus TCU (computer) and a training storage unit or apparatus TMU (memory or database). Therein, the training apparatus TRS can advantageously be configured to carry out the above described computer-implemented method according to the embodiment for providing a trained model for identifying and classifying image features, and/or for providing a trained model for classifying patient-specific image features and/or for providing a trained model for generating synthetic medical image data and/or for providing a further trained model for generating synthetic medical image data and/or for providing a further trained model for classifying patient-specific image features, and their aspects. The training apparatus TRS can be configured to carry out these methods and their aspects in that the training interface TIF and the training computing apparatus TCU are configured to carry out the corresponding method acts. In particular, the training interface TIF can be configured for receiving medical training image data TBD and/or classified training image features and/or synthetic medical training image data TSBD. Additionally, the training interface TIF can be configured to provide the trained model.

The provision apparatus PRVS and/or the training apparatus TRS can be, in particular, a computer, a microcontroller or an integrated circuit. Alternatively, the provision apparatus PRVS and/or the training apparatus TRS can be a real or virtual grouping of computers (a technical term for a real grouping being a “cluster” or, in the case of a virtual grouping, a “cloud”). The provision apparatus PRVS and/or the training apparatus TRS can also be configured as a virtual system which is executed on a real computer or a real or virtual grouping of computers (a technical term therefor being “virtualization”).

An interface IF and/or a training interface TIF can be a hardware or software interface (for example, a PCI bus, USB or Firewire). A computing apparatus CU and/or a training computing apparatus TCU can have hardware elements or software elements, for example, a microprocessor or a so-called FPGA (Field Programmable Gate Array). A storage apparatus MU and/or a training storage apparatus TMU can be realized as a non-permanent working memory (Random Access Memory, (RAM)) or as a permanent mass storage apparatus (hard disk, USB stick, SD card, solid state disk).

The interface IF and/or the training interface TIF can include, in particular, a plurality of subsidiary interfaces that carry out the different acts of the respective method. In other words, the interface IF and/or the training interface TIF can also be regarded as a large number of interfaces IF or a large number of training interfaces TIF. The computing apparatus CU and/or the training computing apparatus TCU can include, in particular, a plurality of subsidiary computing apparatuses which carry out the different acts of the respective method. In other words, the computing apparatus CU and/or the training computing apparatus TCU can also be regarded as a large number of computing apparatuses CU or a large number of training computing apparatuses TCU.

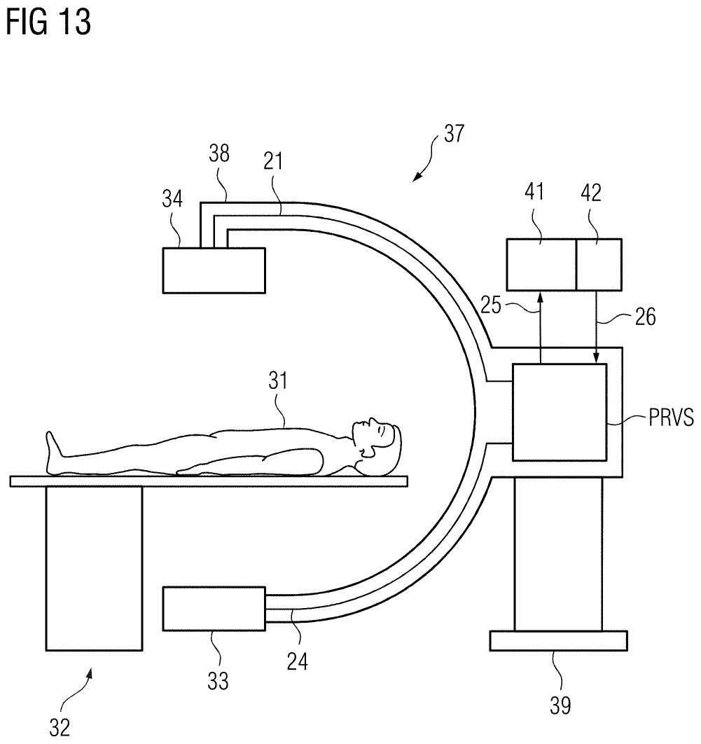

FIG. 13 is a schematic representation of a medical C-arm X-ray device 37 as an example of a proposed medical imaging device. Therein, the medical C-arm X-ray device 37 can advantageously include a proposed provision apparatus PRVS for providing classified image features and/or for providing synthetic medical image data. Therein, the medical imaging device 37, in particular, the proposed provision apparatus PRVS is configured for carrying out a proposed computer-implemented method for providing classified image features and/or for providing synthetic medical image data.

Herein, the medical C-arm X-ray device 37 also includes a detector 34 and an X-ray source 33. For recording medical image data BD, in particular, at least one projection X-ray image, the arm 38 of the C-arm X-ray device 37 can be mounted such that it can move about one or more axes. Additionally, the medical C-arm X-ray device 37 can include a movement apparatus 39, which enables a movement of the C-arm X-ray device 37 in space.

For recording the medical image data BD from an examination region to be mapped of an examination object 31 arranged on a patient positioning apparatus 32, the provision apparatus PRVS can transmit a signal 24 to the X-ray source 33. Thereupon, the X-ray source 33 can emit an X-ray beam, in particular, a conical beam and/or a fan beam and/or a parallel beam. When the X-ray beam impinges upon a surface of the detector 34 following an interaction with the region of the examination object 31 to be mapped, the detector 34 can emit a signal 21 to the provision apparatus PRVS. The provision apparatus PRVS can receive the medical image data BD, for example, based upon the signal 21.

Furthermore, the medical C-arm X-ray device 37 can include an input 41, for example, a keyboard and/or a display screen 42, for example, a monitor and/or display. The input 41 can preferably be integrated into the display 42, for example, in the case of a capacitive input display. Therein, by an input by an operating person to the input 41, a control of the medical C-arm X-ray device 37 can be enabled. For example, a graphical representation of the medical image data BD and/or the classified image features and/or the synthetic medical image data SBD can be displayed on the display 42.

The schematic representations contained in the figures described do not show any scale or size relation.

Finally, it should again be noted that the methods described above in detail and the apparatuses illustrated are merely exemplary embodiments which can be modified by a person skilled in the art in a wide variety of ways without departing from the scope of the invention. Furthermore, the use of the indefinite article “a” or “an” does not preclude the possibility that the relevant features can also be present plurally. Similarly, the expressions “unit” and “element” do not preclude the components in question consisting of a plurality of cooperating subcomponents with can also be spatially distributed. It is therefore intended that the foregoing description be regarded as illustrative rather than limiting, and that it be understood that all equivalents and/or combinations of embodiments are intended to be included in this description.

It is to be understood that the elements and features recited in the appended claims may be combined in different ways to produce new claims that likewise fall within the scope of the present invention. Thus, whereas the dependent claims appended below depend from only a single independent or dependent claim, it is to be understood that these dependent claims can, alternatively, be made to depend in the alternative from any preceding or following claim, whether independent or dependent, and that such new combinations are to be understood as forming a part of the present specification.

1. A computer-implemented method for providing classified image features, the method comprising:
receiving medical image data,
identifying a plurality of image features in the medical image data, and
classifying the plurality of image features into patient-specific image features and non-patient-specific image features by application to first input data of a first trained model for identifying and classifying image features,
wherein the first input data is based upon the medical image data,
wherein at least one parameter of the first trained model for identifying and classifying image features is based upon a comparison of training identification parameters with comparative identification parameters and a comparison of training diagnostic parameters with comparative diagnostic parameters,
providing the classified image features.

2. The computer-implemented method as claimed in claim 1, further comprising:
classifying the patient-specific image features into phenotypically expressed patient-specific image features and into non-phenotypically expressed patient-specific image features by application to second input data a second trained model for classifying patient-specific image features,
wherein the second input data is based upon the patient-specific image features,
wherein at least one parameter of the second trained model for classifying patient-specific image features is based upon a comparison of phenotypically expressed patient-specific training image features with phenotypically expressed patient-specific comparative image features and a comparison of non-phenotypically expressed patient-specific training image features with non-phenotypically expressed patient-specific comparative image features,
providing the classified patient-specific image features.

3. The computer-implemented method as claimed in claim 1, further comprising:
generating synthetic medical image data by application to third input data a third trained model for generating synthetic medical image data,
wherein the third input data is based upon the patient-specific image features,
wherein at least one parameter of the third trained model for generating synthetic medical image data is based upon a comparison of synthetic medical training image data with synthetic medical comparative image data,
providing the synthetic medical image data.

4. The computer-implemented method as claimed in claim 1, further comprising:
generating synthetic medical image data by application to second input data a further trained model for generating the synthetic medical image data,
wherein the second input data is based upon the non-patient-specific image features,
wherein at least one parameter of the further trained model for generating synthetic medical image data is based upon a comparison of synthetic medical training image data with synthetic medical comparative image data,
providing the synthetic medical image data.

5. The computer-implemented method as claimed in claim 2, further comprising:
generating synthetic medical image data by application to third input data a further trained model for generating the synthetic medical image data,
wherein the third input data is based upon the non-phenotypically expressed non-patient-specific image features or the third input data is based upon the non-phenotypically expressed patient-specific image features and the non-patient-specific image features,
wherein at least one parameter of the further trained model for generating synthetic medical image data is based upon a comparison of synthetic medical training image data with synthetic medical comparative image data,
providing the synthetic medical image data.

6. The computer-implemented method as claimed in claim 3, further comprising:
classifying the patient-specific image features into phenotypically expressed patient-specific image features and into non-phenotypically expressed patient-specific image features by application to fourth input data a further trained model for classifying patient-specific image features,
wherein the fourth input data is based upon the patient-specific image features and the synthetic medical image data,
wherein at least one parameter of the further trained model for classifying patient-specific image features is based upon a comparison of the phenotypically expressed patient-specific training image features with the phenotypically expressed patient-specific comparative image features and a comparison of the non-phenotypically expressed patient-specific training image features with the non-phenotypically expressed patient-specific comparative image features,
providing the classified patient-specific image features.

7. A computer-implemented method for identifying and classifying image features, the method comprising:
receiving medical training image data of a plurality of examination objects,
identifying a plurality of training image features in the medical image data, and
classifying the plurality of training image features into patient-specific training image features and non-patient-specific training image features by application to first input data a first trained model for identifying and classifying image features,
wherein the first input data is based upon the medical training image data (TBD),
determining training identification parameters and training diagnostic parameters based upon the classified training image features,
wherein one of the training identification parameters and one of the training diagnostic parameters are determined for each of the classified training image features and/or for a combination of classified training image features,
receiving a comparative identification parameter and a comparative diagnostic parameter for each of the examination objects,
wherein each comparative identification parameter comprises an identification information item relating to one of the examination objects,
wherein each comparative diagnostic parameter comprises a diagnostic information item relating to one of the examination objects,
adapting at least one parameter of the first trained model for identifying and classifying image features based upon a comparison of the training identification parameters with the comparative identification parameters and a comparison of the training diagnostic parameters with the comparative diagnostic parameters,
providing the trained model for identifying and classifying image features.

8. The computer-implemented method of claim 1, further comprising:
providing a second trained model for classifying patient-specific image features, the providing comprising:
receiving medical training image data of a plurality of examination objects, receiving classified training image features,
wherein the classified image features are provided as the patient-specific training image features and non-patient-specific training image features and the patient-specific image features are provided as patient-specific training image features,
classifying the patient-specific training image features, by application of an identification function to the patient-specific training image features, into phenotypically expressed patient-specific comparative image features and non-phenotypically expressed patient-specific comparative image features,
classifying the patient-specific training image features, by application to second input data the second trained model for classifying patient-specific image features, into phenotypically expressed patient-specific training image features and into non-phenotypically expressed patient-specific training image features,
wherein the second input data is based upon the patient-specific training image features,
adapting at least one parameter of the second trained model for classifying patient-specific image features based upon a comparison of the phenotypically expressed patient-specific training image features with the phenotypically expressed patient-specific comparative image features and a comparison of the non-phenotypically expressed patient-specific training image features with the non-phenotypically expressed patient-specific comparative image features.

9. The computer-implemented method of claim 8 wherein the identification function comprises a biometric function.

10. The computer-implemented method of claim 1, further comprising:
providing a third trained model for generating synthetic medical image data, the providing comprising:
receiving medical training image data of a plurality of examination objects,
receiving classified training image features as patient-specific training image features and non-patient-specific training image features wherein the patient-specific image features comprise patient-specific training image features,
generating synthetic medical comparative image data by application of a reconstruction function to the patient-specific training image features,
generating synthetic medical training image data by applying to third input data to the third trained model for generating synthetic medical image data,
wherein the third input data is based upon the patient-specific training image features,
adapting at least one parameter of the third trained model for generating synthetic medical image data based upon a comparison of the synthetic medical comparative image data with the synthetic medical training image data.

11. The computer-implemented method of claim 1 further comprising:
providing a further trained model for generating synthetic medical image data, the providing comprising:
receiving medical training image data of a plurality of examination objects,
receiving classified training image features,
wherein the classified image features are provided as the classified training image features, the non-patient-specific image features are provided as the non-patient-specific training image features,
generating synthetic medical comparative image data by application of a reconstruction function to the non-patient-specific training image features,
generating synthetic medical training image data by application to second input data the further trained model for generating synthetic medical image data,
wherein the second input data is based upon the non-patient-specific training image features,
adapting at least one parameter of the further trained model for generating synthetic medical image data based upon a comparison of the synthetic medical comparative image data with the synthetic medical training image data.

12. The computer-implemented method of claim 2 further comprising:
providing a further trained model for generating synthetic medical image data, the providing comprising:
receiving medical training image data of a plurality of examination objects,
receiving classified training image features,
wherein the classified image features are provided as the classified training image features, the non-phenotypically expressed patient-specific image features are provided as non-phenotypically expressed training image features,
generating synthetic medical comparative image data by application of a reconstruction function to the non-phenotypically expressed patient-specific training image features,
generating synthetic medical training image data by application to third input data the further trained model for generating synthetic medical image data,
wherein the third input data is based upon the non-phenotypically expressed patient-specific training image features or the third input data is based upon the non-phenotypically expressed patient-specific training image features and the non-patient-specific training image features,
adapting at least one parameter of the further trained model for generating synthetic medical image data based upon a comparison of the synthetic medical comparative image data with the synthetic medical training image data.

13. The computer-implemented method of claim 3 further comprising:
providing a further trained model for classifying patient-specific image features, the providing comprising:
receiving medical training image data of a plurality of examination objects,
receiving the synthetic medical training image data, wherein the synthetic medical image data is provided as the synthetic medical training image data and the patient-specific image features are provided as the patient-specific training image features,
classifying the patient-specific training image features, by application of a further identification function to the patient-specific training image features and the synthetic medical training image features, into phenotypically expressed patient-specific comparative image features and non-phenotypically expressed patient-specific comparative image features,
classifying the patient-specific training image features by application to fourth input data the further trained model for classifying patient-specific image features into phenotypically expressed patient-specific training image features and into non-phenotypically expressed patient-specific training image features,
wherein the fourth input data is based upon the patient-specific training image features and the synthetic medical training image data,
adapting at least one parameter of the further trained model for classifying patient-specific image features based upon a comparison of the phenotypically expressed patient-specific training image features with the phenotypically expressed patient-specific comparative image features and a comparison of the non-phenotypically expressed patient-specific training image features with the non-phenotypically expressed patient-specific comparative image features.

14. The computer-implemented method of claim 13 wherein the further identification function comprises a biometric function.

15. A provision apparatus for providing classified image features, the provision apparatus comprising:
a computer and an interface,
wherein the interface is configured for receiving medical image data,
wherein the computer is configured for identifying a plurality of image features in the medical image data and classifying the plurality of image features into patient-specific image features and non-patient-specific image features by application to first input data a first trained model for identifying and classifying image features,
wherein the first input data is based upon the medical image data,
wherein at least one parameter of the first trained model for identifying and classifying image features is based upon a comparison of training identification parameters with comparative identification parameters and a comparison of training diagnostic parameters with comparative diagnostic parameters,
wherein the interface is further configured for providing the classified image features.

16. The provision apparatus of claim 15, wherein the interface is configured to provide synthetic medical image data,
wherein the interface is further configured for receiving the patient-specific image features, the non-patient-specific image features, phenotypically expressed patient-specific image features, and non-phenotypically expressed patient-specific image features,
wherein the computer is configured to generate the synthetic medical image data by application to second input data a second trained model for generating the synthetic medical image data,
wherein the second input data is based upon the patient-specific image features,
wherein at least one parameter of the second trained model for generating the synthetic medical image data is based upon a comparison of the synthetic medical training image data with synthetic medical comparative image data (SVBD).
